(19) United States
(12) Patent Application Publication
(10) Pub. No.: US 2025/0075248 A1
(43) Pub. Date: Mar. 6, 2025

(54) SYSTEMS, METHODS, AND DEVICES FOR ANTIMICROBIAL SUSCEPTIBILITY TESTING

(71) Applicant: Deepull Diagnostics S.L., Barcelona (ES)

(72) Inventors: Rafael BRU GIBERT, Barcelona (ES); Jordi CARRERA FABRA, Barcelona (ES); Marta MARÍ ALMIRALL, Barcelona (ES); Ariadna SANGLAS BAULENAS, Sabadell (ES)

(73) Assignee: Deepull Diagnostics S.L., Barcelona (ES)

(21) Appl. No.: 18/728,766

(22) PCT Filed: Jan. 13, 2023
(86) PCT No.: PCT/IB2023/050337

§ 371 Date: Jul. 12, 2024

Related U.S. Application Data
(60) Provisional application No. 63/299,555, filed on Jan. 14, 2022.

(51) Int. Cl. C12Q 1/18; B01L 3/00

(52) U.S. Cl. CPC C12Q 1/18; B01L 3/50851; B01L 2300/0829; B01L 2400/043

(57) ABSTRACT

Described herein are systems, methods, and devices for antimicrobial susceptibility testing (AST) directly from blood samples. A system for enriching samples includes a housing configured to receive sample containers, sample preparation cartridges, and AST cartridges, for processing and testing samples using one or more centrifuges, pipetting systems, a controller, AST subsystem, magnet station, microscope, and heater disposed inside the housing. The system is configured to transfer a sample from a blood sample container into a processing tube in a sample preparation cartridge using a septum and needle-based pipetting system. Upon transfer of the sample, the system performs steps to separate, enrich, and concentrate pathogens in the sample for rapid detection. The system then dispenses aliquots of the enriched sample into antimicrobial-containing reaction wells in an AST cartridge, acquires images of each reaction well after incubation, and determines pathogen susceptibility to antimicrobials based on analyzing the acquired images.

### CROSS-REFERENCE TO RELATED APPLICATIONS

This application claims the benefit of U.S. Provisional Application No. 63/299,555, filed Jan. 14, 2022, which is incorporated herein by reference in its entirety. This application is related to co-pending U.S. patent application Ser. No. ______ by Bru Gibert et al., entitled “Systems, Methods, and Devices for Pathogen Identification” filed on Jan. 13, 2023 (Atty Docket No. 4518.003PC01), and PCT Application No. PCT/US2022/039290, filed on Aug. 3, 2022, the disclosures of which are incorporated by reference herein in their entireties.

### BACKGROUND

### Field

Embodiments of the present disclosure relate to systems, methods, and devices for performing antimicrobial susceptibility testing (AST) from whole blood or other samples for determining treatment for patients.

### Background

Sepsis is defined as life-threatening organ dysfunction caused by a dysregulated host response to infection. A patient experiencing or undergoing sepsis may start with a local infection, such as pneumonia, that results in inflammation of the body from the patient's immune system going into overdrive. Inflammation may ultimately lead to organ failure and death of the patient if left untreated. Sepsis causes 11 million deaths annually, and many of these cases can be prevented by early diagnosis, proper clinical management, and treatment.

Sepsis diagnosis may be performed by running laboratory tests to culture blood samples for identifying infection. Current testing times may take several days to obtain results from a laboratory as a result of the time needed for obtaining and processing blood cultures for sepsis-causing bacteria to ultimately determine its susceptibility to effective antimicrobials for treatment. However, sepsis detection may be time-sensitive for patients in a hospital, because sepsis can lead to septic shock and death within hours if not properly identified and treated in time. Additionally, blood culture testing is slow and might not consistently provide reliable results of bacteria or fungi detection in patients who are clinically suspected of having sepsis, especially for patients already undergoing antibiotic therapy. There is often a low yield for obtaining positive blood cultures for patients, and patients may be experiencing sepsis even without the identification of a positive blood culture.

Traditional and automated methods for performing susceptibility testing of antimicrobials necessitate a “pure culture” of the bacterial isolate, along with a prolonged period (e.g., 6-24 hours) of incubation for growth of the organism. Such conventional systems may be limited because they are growth-based, slow, expensive, necessitate manual manipulation, and are not integrated. Without improved solutions, patients may continue to suffer and often becomes worse as physicians treat with empiric antibiotics while awaiting more actionable information from the laboratories about the one or more causative agents for the infection (e.g., pathogens) and with which antimicrobials to treat highly resistant pathogens in patients undergoing infection.

### BRIEF SUMMARY

Embodiments of the present disclosure provide cost-effective solutions for improved diagnostic methods, systems, and devices for antimicrobial susceptibility testing in order to provide appropriate treatments to patients for better patient outcomes.

Described herein are systems, methods, and devices for performing antimicrobial susceptibility testing (AST) directly from blood samples, or other samples, such as urine, sterile body fluids, or the like. In the embodiments presented herein, AST systems, analyzer devices, AST cartridges, sample preparation cartridges, and processing tubes are provided to perform rapid AST from samples without blood culture, while preserving specimen quality of samples. In some embodiments, single cell microscopy may be leveraged to identify phenotypical susceptibility of the pathogen, leading to a diagnostic pathway for rapid and effective antimicrobial treatments. In some embodiments, the systems, methods, and devices for AST described herein may be used to treat patients with sepsis and/or other underlying diseases.

In an embodiment, an example method for performing susceptibility testing is described. The method includes receiving, by an analyzer device, a sample preparation cartridge and a sample container, the sample container containing a sample comprising pathogens, installing a first needle from the sample preparation cartridge in a pipettor system in the analyzer device, inserting the first needle into the sample container using the pipettor system, transferring the sample from the sample container through the first needle to a processing tube in the sample preparation cartridge, concentrating and enriching the pathogens of the sample in the processing tube using the analyzer device, resulting in an enriched sample in the processing tube, and dispensing a plurality of aliquots of the enriched sample to a plurality of reaction wells in an antimicrobial susceptibility testing (AST) cartridge in the analyzer device, wherein each aliquot corresponds to a respective reaction well, and wherein each reaction well comprises an antimicrobial of a predetermined concentration. The method further includes incubating the aliquots in the reaction wells of the AST cartridge for a predetermined period of time for a reaction to occur between the pathogens in the aliquots and the antimicrobial in each reaction well, acquiring an image of each reaction well in the AST cartridge by using a microscope in the analyzer device, and determining, by a processor coupled to the microscope in the analyzer device, a susceptibility of the pathogens to the antimicrobial in each reaction well by analyzing the image.

In another embodiment, an example antimicrobial susceptibility testing (AST) cartridge is described. The AST cartridge includes a base comprising a plurality of reaction wells, each reaction well comprising a bottom wall, in which the bottom wall is optically transparent. The AST cartridge further includes a septum disposed over the base, the septum sealing each reaction well in the plurality of reaction wells, and a cover disposed over the septum. Each reaction well in the plurality of reaction wells contains an antimicrobial of a predetermined concentration for reacting with a respective aliquot of an enriched sample comprising pathogens, and the antimicrobial is disposed within each reaction well.

In another embodiment, an example system for enriching samples is described. The system includes a housing configured to receive a sample container containing a sample comprising pathogens, a pipettor system disposed inside the housing, one or more centrifuges disposed inside the housing, and a controller. The controller is configured to transfer the sample from the sample container to a processing tube using the pipettor system, centrifuge the processing tube using the one or more centrifuges to concentrate the pathogens in the sample, remove a fluid from the processing tube using the pipettor system, leaving the concentrated pathogens in the processing tube, and add a growth media to the concentrated pathogens in the processing tube using the pipettor system to grow the concentrated pathogens in the processing tube for a predetermined period of time, and clean the concentrated pathogens after the predetermined period of time to obtain an enriched sample in the processing tube.

In another embodiment, an example system for analyzing samples is described. The system includes a housing configured to receive a processing tube and an antimicrobial susceptibility testing (AST) cartridge. The AST cartridge includes a plurality of reaction wells, each reaction well comprising a bottom wall, in which the bottom wall is optically transparent. The system further includes a heater disposed inside the housing, a pipettor system disposed inside the housing, a microscope disposed inside the housing, and a controller. The controller is configured to dispense, using the pipettor system, a plurality of aliquots of an enriched sample comprising pathogens from the processing tube to the plurality of reaction wells in the AST cartridge, in which each aliquot corresponds to a respective reaction well, and each reaction well comprises an antimicrobial of a predetermined concentration. The controller is further configured to incubate, using the heater, the aliquots in the reaction wells of the AST cartridge for a predetermined period of time for a reaction to occur between the pathogens and the antimicrobial in each reaction well, acquire, using the microscope, one or more images of a bottom wall of each reaction well in the AST cartridge, and determine, by a processor coupled to the microscope, a susceptibility of the pathogens to the antimicrobial in each respective reaction well by analyzing the one or more images.

In another embodiment, an example method for manufacturing an AST cartridge is described. The method includes fabricating a cover comprising a plurality of openings, overmolding a septum into the cover, wherein a first side of the septum extends across the plurality of openings, producing a base comprising a plurality of reaction wells, and attaching the base to a second side of the septum, wherein the second side of the septum extends across and seals the plurality of reaction wells in the base.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form a part of the specification, illustrate embodiments of the present disclosure and, together with the description, further serve to explain the principles of the disclosure and to enable a person skilled in the pertinent art to make and use the disclosure.

FIG. 1 illustrates a diagram of a system for performing antimicrobial susceptibility testing (AST), according to embodiments of the present disclosure.

FIG. 2 illustrates a diagram of an analyzer device, according to embodiments of the present disclosure.

FIG. 3A illustrates a diagram of a front view of an analyzer device, according to embodiments of the present disclosure.

FIG. 3B illustrates a diagram of a top view of an analyzer device, according to embodiments of the present disclosure.

FIG. 4 illustrates a diagram of an analyzer device with three opening compartments, according to embodiments of the present disclosure.

FIG. 5 illustrates a diagram of a sample preparation cartridge, according to embodiments of the present disclosure.

FIG. 6A illustrates a diagram of a processing tube and other components within the sample preparation cartridge, according to embodiments of the present disclosure.

FIG. 6B illustrates a diagram of a processing tube and other components within a linear sample preparation cartridge, according to embodiments of the present disclosure.

FIGS. 7A and 7B illustrate diagrams of a processing tube, according to embodiments of the present disclosure.

FIGS. 8A, 8B, and 8C illustrate diagrams of a needle configured for insertion into a processing tube, according to embodiments of the present disclosure.

FIGS. 9A and 9B illustrate diagrams of a high volume needle and a low volume needle, respectively, according to embodiments of the present disclosure.

FIG. 10 illustrates a diagram of examples of a high volume needle, according to embodiments of the present disclosure.

FIG. 11 illustrates a diagram of a low volume needle interfacing with a sample preparation cartridge, according to embodiments of the present disclosure.

FIGS. 12A and 12B illustrate diagrams of an AST cartridge, according to embodiments of the present disclosure.

FIG. 13 illustrates a diagram of a low volume needle being inserted into an AST cartridge, according to embodiments of the present disclosure.

FIGS. 14A and 14B illustrate diagrams of example centrifuges used in an analyzer, according to embodiments of the present disclosure.

FIGS. 15A, 15B, 15C, and 15D illustrate diagrams of an example enrichment subsystem used in an analyzer, according to embodiments of the present disclosure.

FIGS. 16A, 16B, and 16C illustrate diagrams of an example mechanical apparatus used in an analyzer, according to embodiments of the present disclosure.

FIGS. 17A and 17B illustrate diagrams of an AST cartridge interfacing with an imaging subsystem in the analyzer, according to embodiments of the present disclosure.

FIG. 18 illustrates a flowchart diagram of a method for performing AST of a sample, according to embodiments of the present disclosure.

FIG. 19 illustrates a flowchart diagram of a method for manufacturing or assembling an AST cartridge, according to embodiments of the present disclosure.

FIG. 20 illustrates a block diagram of example components of a computer system, according to embodiments of the present disclosure.

FIGS. 21-27 illustrate experimental results from tests based on embodiments of the present disclosure.

Embodiments of the present disclosure will be described with reference to the accompanying drawings.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that this disclosure can also be employed in a variety of other applications.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

### INTRODUCTION

The current standard of care for detecting and treating sepsis relies on blood culture, for which the average time to detection is about 13 hours. Blood culture testing supplies an organism, without identification (ID) of the pathogen, followed by plating of positives on petri dishes. In a conventional blood culture process, two blood culture sets are taken per adult patient, in which each set consists of an aerobic bottle and an anaerobic bottle to assure that the entire spectrum of sepsis causative bacteria is captured during the culture event. Generally, each culture is acquired from a separate venipuncture (e.g., left arm and right arm of the patient). This is to assure that the bacterial shedding event is captured by the culture so that the bacteria may be “recovered” for downstream testing (e.g., ID and AST). Following the culturing, the aerobic and anaerobic bottles are incubated in a blood culture instrument where they are monitored in real-time for growth. The aerobic and anaerobic bottles are incubated and agitated until any bacteria is allowed to go through a lag-log growth transition that is detected electronically. A laboratory worker may then be alerted that a positive culture exists for the patient. Typically, a blood culture will become positive in an average of about 13 hours for most bacteria, while some yeasts and fungi may take much longer (e.g., up to 5 days). However, many cultures are negative due to collection error, an insufficient blood volume taken during collection, transport delays to the lab, insufficient sensitivity, or the like.

Due to the urgent nature of sepsis, following positivity, a laboratory may immediately commence a work-up to identify the bacterial gram stain (e.g., gram positive or gram negative), determine a significant organism, contaminant, single microbe, or polymicrobic infection, and report the intermediate information to the caregiver. Further, the lab may take immediate steps to identify the bacteria using rapid methods such as molecular diagnostics systems, which may take 1.5 hours to provide results. These systems may offer limited molecular information on genetic drug resistance information of certain bacteria that exhibit these profiles. Alternatively, the lab may process the positive blood culture (PBC) aliquot with a matrix-assisted laser desorption/ionization time of flight (MALDI-TOF) mass spectrometry system to report an ID in about one hour. Using the ID, the caregiver may confirm or potentially adjust antibiotics that may have been administered to the patient prophylactically. However, by the time the bacteria has been identified, up to 20-24 hours (at best) may have already passed since the patient was first cultured.

In parallel to obtaining and reporting the identified pathogen, antimicrobial susceptibility testing (AST) may also be performed to determine the antibiotic susceptibility profile of the pathogen. In some cases, AST may be performed in vitro on an isolated bacterial pathogen using manual methods including microbroth dilution methods or culture-based assays (e.g., disc-diffusion antibiotic susceptibility test or using plated petri dish media). In other cases, AST may be performed using automated devices with antimicrobial resistance panels to test for minimum inhibitory concentration (MIC) of antimicrobials or drug resistance.

However, these systems may require isolated bacterial colonies. For example, current methods for AST may necessitate a significant biomass of clean bacteria to operate properly. The subculture from the positive blood culture first has to be grown out on a plated media petri dish. This process may take 6-12 hours for a round-the-clock operating lab, but may sometimes take up to 24 hours for a lab that is closed overnight. The culture may then need to be adjusted for uniformity (e.g., 10,000 cells taken for input) before being inoculated into the AST system. Following loading, the AST analysis can take between 8-16 hours to report antimicrobial susceptibility or resistance information for all organism classes. Additionally, AST might not begin until at least day 2 of a patient's stay in the hospital. In other words, results from the AST analysis (e.g., including whether the bacteria is susceptible, intermediate, or resistant (SIR) to particular antibiotics, MIC, or resistance information) may be reported to the caregiver about 2.5-3 days after an initial collection of the original blood culture sample from the patient.

The overall process may take several days to report critical antibiotic information for sepsis patients. For example, if an infection is suspected, a sample of blood, urine, sputum, or the like, is collected from the patient and provided to a clinical laboratory to first determine if an infectious agent is present. This may require 18-24 hours (e.g., day 1) for most pathogenic bacterial species to sufficiently grow. If a bacterium is isolated, it further requires an additional 18-24 hours (e.g., day 2) to culture the isolate and another 2-48 hours (e.g., day 3+) to identify the bacterial isolate and perform AST. Traditional and automated AST methods require a “pure culture” of the bacterial isolate, along with a prolonged period (e.g., 6-24 hours) of incubation for growth of the organism. Conventional systems may be limited because they are growth-based, slow, expensive, require manual manipulation, and are not integrated. Without improved solutions, patients may continue to suffer and often become worse as physicians treat with empiric antibiotics while awaiting more actionable information from the laboratories about the one or more causative agents for the infection (e.g., pathogens).

Current technologies do not provide an integrated and comprehensive solution for the entire workflow of host response detection, pathogen identification, and antimicrobial or antibiotic susceptibility testing (AST). In some cases, some systems focus solely on identifying a single aspect of the sepsis cascade, such as detection of host response or detection of a pathogen. For example, a system may look at host response for early indication of sepsis by detecting molecular white cell RNA markers (via reverse transcription (RT)-PCR to detect gene expression), but would not provide answers for pathogen identification or susceptibility. The immune response result may alert caregivers that a patient is entering or has entered the sepsis cascade and is in urgent need of treatment or intervention to prevent further probability of irreversible morbidity. Caregivers may immediately react by looking for the infection site and the infectious agent using traditional methods, such as by obtaining blood cultures from the infection site to identify the infectious agent.

On the pathogen detection side, current technologies may offer a direct from blood detection and identification method, using PCR from blood samples and followed by detection. However, such systems may be expensive, limited in menu options, and difficult to service without providing a solution for rapid AST results, instead offering a limited molecular genetic resistance panel. Other systems may utilize direct-from-blood pathogen rRNA RT-PCR for pathogen identification but may also be constrained by a limited menu (e.g., 15 targets or less). Ultimately, current technologies do not provide an automated direct-from-blood rapid AST solution, and instead rely on positive blood cultures for testing which can take 13-20 hours to report anything actionable. For example, some systems may obtain aliquots from positive blood culture (PBC) bottles, thus saving the time needed to grow the bacterial isolate from the PBC bottle (e.g., 6-24 hours). These systems, however, are limited as to the numbers of drugs and organisms they can report on and thus have limited utility for healthcare providers. In order to greatly reduce morbidity and mortality, new diagnostic methods, devices, and systems are needed for rapidly determining antibiotic sensitivity and antibiotic resistance to an infectious sepsis-causing bacteria at the single cell or low copy number level, which is detected directly from a blood sample without the significant time delay for the multiple culture steps (e.g., biological amplification) currently necessitated by the standard of care methods.

As septic conditions may often go undetected in patients and quickly progress into life-threatening conditions, there is a clear need and demand for new and comprehensive systems, devices, and methods for performing rapid testing on patient samples and providing guidance on appropriate antimicrobial agents in order to save lives and reduce antimicrobial resistance (AMR). The systems, devices, and methods described herein provide a holistic and systematic approach for determining antibiotic sensitivity and resistance to pathogens in order to recommend treatments that are effective and appropriate for patients.

### AST System Overview:

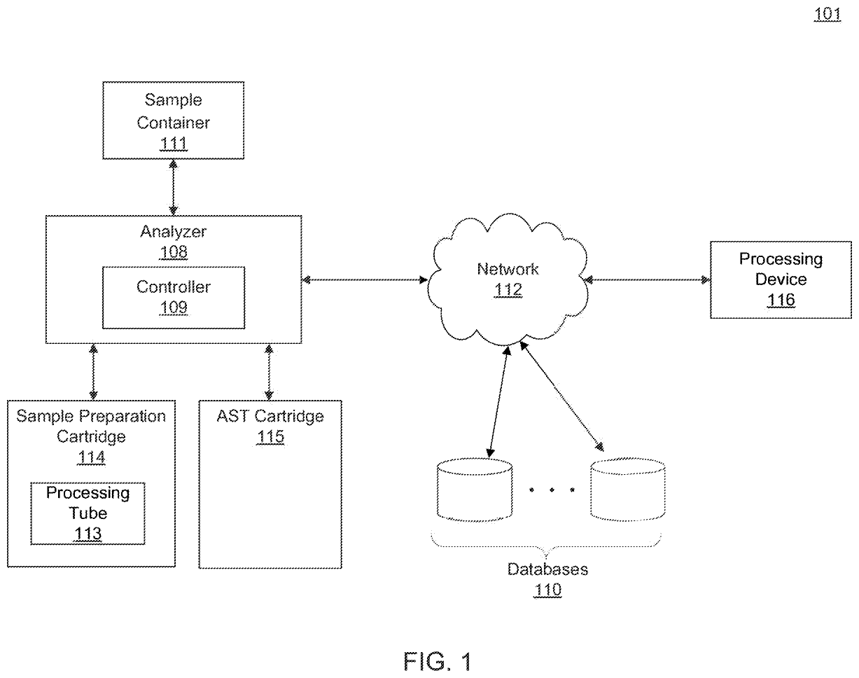

FIG. 1 illustrates a diagram of a system 101 for performing antimicrobial susceptibility testing (AST), according to embodiments of the present disclosure. In some embodiments, the system 101 may be referred to herein as an AST system 101. The system 101 may comprise an analyzer 108, a sample container 111, sample preparation cartridge 114, AST cartridge 115, processing device 116, and a plurality of databases 110 communicatively coupled via a network 112.

The analyzer 108 may be a point-of-care (POC) testing device that performs phenotypic AST of pathogens in a sample of a patient, which may be stored in sample container 111. In some embodiments, the analyzer 108 may be referred to herein as an analyzer device. In some embodiments, the sample in sample container 111 may comprise whole blood, urine, sterile body fluids, or other samples obtained from the patient. In some embodiments, the analyzer 108 may receive the sample container 111, which is placed into a corresponding drawer in a housing of the analyzer 108 by a user or operator of the analyzer 108.

In addition to receiving the sample container 111, the analyzer 108 may also receive the sample preparation cartridge 114 and AST cartridge 115, which may similarly be placed into corresponding drawers of the housing of the analyzer 108 by a user or operator of the analyzer 108. In some embodiments, various components and subsystems in the analyzer 108 may interface with the sample container 111, sample preparation cartridge 114, AST cartridge 115, processing device 116, and/or databases 110 to perform sample preparation and processing, concentration of pathogens in samples followed by enrichment and clean-up, and AST using microscopy and/or fluorescence.

In some embodiments, after receiving the sample container 111, a pipetting system in the analyzer 108 may transfer the sample from the sample container 111 to the sample preparation cartridge 114. In some embodiments, the sample preparation cartridge 114 may be a specialized consumable with receptacles configured to hold the sample and elements for performing sample processing. The sample preparation cartridge 114 may include a processing tube 113 that has a septum disposed over or within the tube for protecting contents therein. The processing tube 113 may be configured to receive the sample from the sample container 111 through the septum, by using a needle of the sample preparation cartridge 114 to transfer the sample. Once the sample is transferred to the processing tube 113, the sample may undergo sample concentration, lysis, enrichment, clean-up, and/or other processing steps.

In some embodiments, the sample container 111 is a blood collection tube. Blood collection tubes are available from different vendors and may include diverse reagents to preserve blood.

In some embodiments, the sample container 111 is a blood culture bottle. Blood culture bottles are available from different vendors and contain a growth medium that encourages microorganisms to multiply. Blood culture bottles may also include resins that absorb antibiotics to reduce their action against the microorganisms in the sample. In some embodiments, the sample in the blood culture bottle is incubated for an amount of time to allow pathogens to grow but not reach the growth plateau (positivity).

After sample preparation and processing (e.g., including concentration, enrichment, and clean-up of the sample) using elements and components of the sample preparation cartridge 114, the sample is transferred from the processing tube 113 in the sample preparation cartridge 114 to the AST cartridge 115 by the pipetting system in the analyzer 108. In some embodiments, the AST cartridge 115 may be a specialized consumable with a plurality of reaction wells configured to hold a plurality of aliquots of an enriched sample for performing AST. In some embodiments, the analyzer 108 may use microscopy (e.g., single cell microscopy, fluorescence microscopy, or the like) by exposing a plurality of aliquots of the enriched sample to predetermined concentrations of antimicrobials in the AST cartridge 115.

In some embodiments, the analyzer 108 may further interface with additional cartridges, such as a polymerase chain reaction (PCR) cartridge. In some embodiments, the PCR cartridge may be a specialized consumable with a plurality of reaction chambers configured to hold a plurality of aliquots of a sample for performing nucleic acid amplification steps for pathogen identification. In some embodiments, the analyzer 108 may be configured to hold one or more sample preparation cartridges 114, PCR cartridges, and/or AST cartridges 115 at a time for performing sample preparation/processing, pathogen identification, and/or susceptibly testing concurrently or consecutively. In some embodiments, the sample preparation cartridge 114, AST cartridge 115, and/or PCR cartridge may be referred to herein as consumables or containers configured for insertion into the analyzer 108.

In some embodiments, the analyzer 108 may include a controller 109 that is disposed inside the housing of the analyzer 108. The controller 109 may control movements and operations of different components within the analyzer 108, including movements of one or more sample containers, processing tubes, cartridges, and pipetting systems in the analyzer 108. In some embodiments, the controller 109 may also control operations of one or more centrifuges, subsystems, and modules in the analyzer 108 for performing sample preparation, pathogen identification, susceptibility testing, and/or other functions. In some embodiments, the controller 109 may include a microcontroller on an integrated circuit (IC) chip in the analyzer 108 that is programmed is turn on/off and operate one or more centrifuges, subsystems, and modules in the analyzer 108. In some embodiments, the controller 109 may be coupled to one or more stepper motors, actuators, or other motion control components in the analyzer 108 and programmed to control movements.

In some embodiments, the controller 109 may be programmed by the processing device 116. The processing device 116 may be a computing device coupled to the analyzer 108 for performing data processing and providing instructions to the controller 109 and/or other components in the analyzer 108. In some embodiments, the processing device 116 may be a personal digital assistant, desktop workstation, laptop or notebook computer, netbook, tablet, smart phone, mobile phone, smart watch, or any combination thereof.

In some embodiments, the processing device 116 may communicate with the analyzer 108 to receive results of the reactions occurring in the AST cartridge 115 and perform further processing and data analysis to determine susceptibility of one or more pathogens of the sample. In some embodiments, the processing device 116 may receive one or more images of the AST cartridge 115 from the analyzer 108 and compares images with a control to determine whether a particular pathogen is susceptible, intermediate, or resistant to a particular antimicrobial.

In some embodiments, the processing device 116 may also communicate with the plurality of databases 110. In some embodiments, one or more of the plurality of databases 110 may represent any number of databases, and may include various databases that store clinical parameters data, epidemiology information or antibiotic resistance information for a plurality of pathogens, or the like. In some embodiments, one or more of the plurality of databases 110 may be configured to store pathogen taxonomy data and/or outcomes from previous pathogen identification workflows (e.g., performed by analyzer 108). In some embodiments, information from one or more databases 110 may be used to select which antimicrobials to test using the AST cartridge 115 and the analyzer 108. In some embodiments, the information from one or more databases 110 may be used to determine, from a result of testing by the AST system 101, whether a tested pathogen is resistant, intermediate or susceptible to a drug. In some embodiments, one or more of the plurality of databases 110 may be configured to store predefined rules for making calls of susceptibility to antimicrobials for various pathogens. In some embodiments, the processing device 116 may use at least one of the pathogen taxonomy data, outcomes, and/or predefined rules in the databases 110 for reporting susceptibility information to a user of the analyzer 108. In some embodiments, the processing device 116 might not report susceptibility of a particular drug to a pathogen based on identifying that the pathogen is not susceptible to the particular drug from parsing the predefined rules. For example, Klebsiella is naturally resistant to ampicillin, so the processing device 116 may not report this susceptibility information to the user.

In some embodiments, one or more of the plurality of databases 110 may comprise electronic health record (EHR) data comprising patient healthcare information obtained from various healthcare services and healthcare providers, such as hospitals, clinical care facilities, laboratories, radiology providers, and pharmacies. In some embodiments, the EHR data stored in the databases 110 may comprise patient data and medical history data regarding the health and treatment of patients, including demographics, medical history, medication and allergies, immunization status, laboratory test results, radiology images, vital signs, personal statistics like age and weight, and billing information for each patient. In some embodiments, the processing device 116 may use results from pathogen identification and/or AST performed by the analyzer 108, along with data stored in the plurality of databases 110 (e.g., clinical parameters data, epidemiology information or antibiotic resistance information, EHR data, or the like) to determine treatment recommendations for patients.

In some embodiments, the components in system 101 may be communicatively coupled via network 112. In particular, the network 112 may allow transmission of information and communication between the analyzer 108, the plurality of databases 110, processing device 116, and/or any other devices or components in the system 101. In some embodiments, the system 101 may include additional components, such as a Raman spectroscopy device and/or electronic health record (EHR) system (not shown).

In some embodiments, network 112 may be any one or any combination of a LAN (local area network), WAN (wide area network), telephone network, wireless network, point-to-point network, star network, token ring network, hub network, or other appropriate configuration. The network may comply with one or more network protocols, including an Institute of Electrical and Electronics Engineers (IEEE) protocol, a 3rd Generation Partnership Project (3GPP) protocol, a 4^{th} generation wireless protocol (4G) (e.g., the Long Term Evolution (LTE) standard, LTE Advanced, LTE Advanced Pro), a fifth generation wireless protocol (5G), and/or similar wired and/or wireless protocols, and may include one or more intermediary devices for routing data between the analyzer 108, the plurality of databases 110, processing device 116, and/or any other devices or components in the system 101.

### Analyzer Device Embodiments:

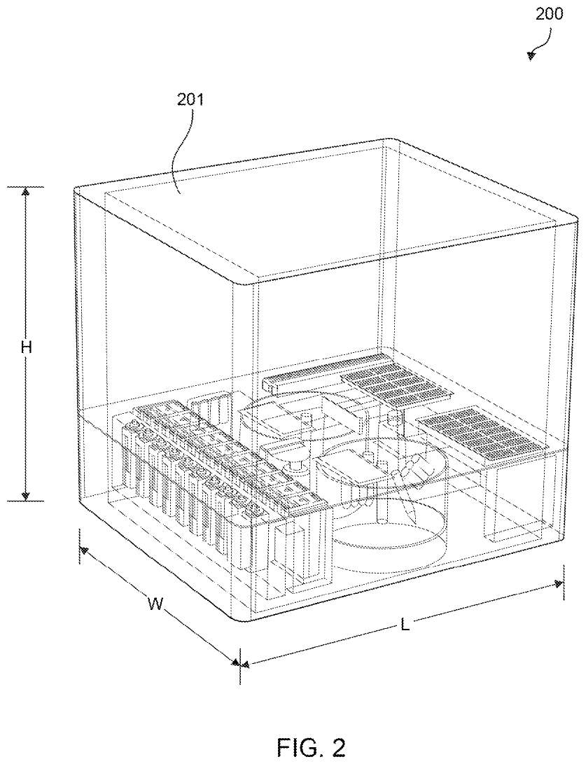

FIG. 2 illustrates a diagram of an analyzer device 200, according to embodiments of the present disclosure. Analyzer device 200 represents an exemplary embodiment of analyzer 108 shown in FIG. 1. In some embodiments, the analyzer device 200 may be referred to herein as an analyzer 200. The analyzer device 200 is a bench-top device with a housing 201, in which various components and modules for performing sample preparation, processing, and testing are housed. In some embodiments, the housing 201 may comprise a body of the analyzer device 200 and/or an exterior case of the analyzer device 200 that protects the modules and components within. In some embodiments, the analyzer device 200 may comprise a cubical, cuboid, or rectangular shape with various compartments for access and operation by a user of the analyzer device 200. In some embodiments, the analyzer device 200 may have a compact size with dimensions of less than about 1 m³, for example about 750 mm (length)×650 mm (width)×650 mm (height).

In some embodiments, the analyzer device 200 may be coupled to a computing device (e.g., processing device 116), such as a personal digital assistant, desktop workstation, laptop or notebook computer, netbook, tablet, smart phone, mobile phone, smart watch, or any combination thereof. A user or operator of the analyzer device 200 may use the computing device to control the analyzer device 200, send/receive sample information, patient information, pathogen information, antimicrobial information, or the like to/from the analyzer device 200, and access/edit results of the susceptibility testing from the analyzer device 200.

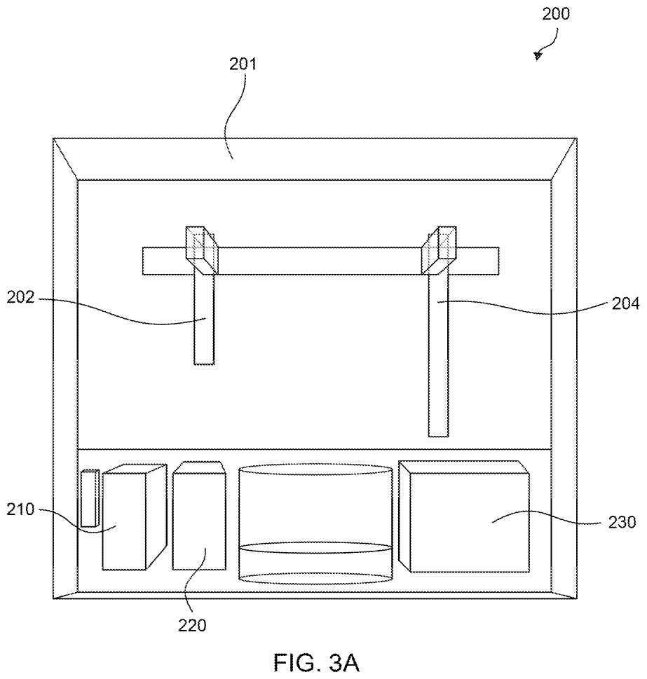

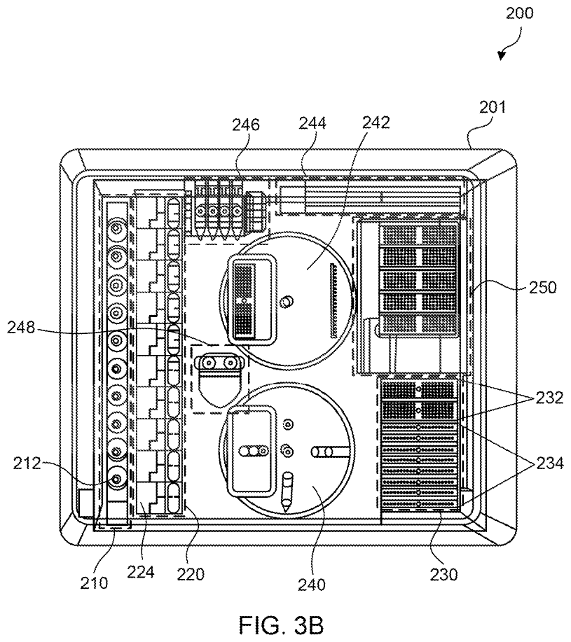

FIG. 3A illustrates a diagram of a front view of the analyzer device 200, according to embodiments of the present disclosure. FIG. 3A illustrates internal features arranged within housing 201 of the analyzer device 200, including first pipettor 202, second pipettor 204, sample drawer 210, sample cartridge drawer 220, and processing cartridge drawer 230.

In some embodiments, the first pipettor 202 and the second pipettor 204 may be pipettor devices configured to handle liquid transfer between components within the housing 201 of the analyzer device 200. In some embodiments, the first and second pipettors 202, 204 may be automated devices that are controlled by the controller 109 in the analyzer device 200. In some embodiments, the controller 109 may control movements of the first and second pipettors 202, 204, including vertical and/or horizontal movements of the first and second pipettors 202, 204 to and/or from different components within the analyzer device 200. In some embodiments, more or fewer pipettors may be present in the analyzer device 200.

In some embodiments, the first pipettor 202 and the second pipettor 204 may be referred to as a high volume pipettor and a low volume pipettor, respectively. In some embodiments, the first pipettor 202 may be configured to handle a volume in a range of about 50 microliters (μL) to 5 milliliters (mL), whereas the second pipettor 204 may be configured to handle a volume in a range of about 1 to 200 μL. In some embodiments, the first pipettor 202 may have a 5% coefficient of variation (CV) at 50 μL and a 1% CV at 5 mL, and the second pipettor 204 may have a 5% CV at 1 to 200 μL.

In some embodiments, the first and second pipettors 202, 204 may be referred to herein as pipettes and/or pipettor systems. In some embodiments, the first and second pipettors 202, 204 may each be configured to attach to needles that are removable and disposable. The first and second pipettors 202, 204 may attach to two different needles configured to handle different volume ranges as necessitated by the first and second pipettors 202, 204.

In addition to liquid transfer, the first and second pipettors 202, 204 may be configured to move elements, such as tubes, cartridges, or the like, within the housing 201 of the analyzer device 200. In particular, the first and second pipettors 202, 204 may each comprise a pipette tip holder that may attach to various components used in the analyzer device 200. In particular, the pipette tip of the first and second pipettors 202, 204 may press and fit into handling features of needles, tubes, cartridges, spin column baskets, and the like, to pick up various components and place them in different modules or areas in the analyzer device 200.

In some embodiments, the dimensions of the first and second pipettors 202, 204 may be about 325 mm (width)×575 mm (depth)×435 mm (height). In some embodiments, the first and second pipettors 202, 204 may be arranged in a top section of the housing 201, such that the first and second pipettors 202, 204 may interact with the samples, tubes, cartridges, and different modules in the bottom section of the housing 201. In particular, the first and second pipettors 202, 204 may handle liquid transfer and movement of components in the sample drawer 210, sample cartridge drawer 220, and processing cartridge drawer 230 shown in FIG. 3A.

In some embodiments, the sample drawer 210, sample cartridge drawer 220, and processing cartridge drawer 230 comprise sliding horizontal compartments that are designed to fit within three corresponding receptacles in the housing 201 of the analyzer device 200. In some embodiments, the sample drawer 210, sample cartridge drawer 220, and the processing cartridge drawer 230 may be configured to receive specialized elements that are inserted into the analyzer device 200 for sample processing and testing. In particular, the sample drawer 210 may receive a sample container (e.g., sample container 111) containing a sample obtained from a patient. The sample cartridge drawer 220 may receive a sample preparation cartridge (e.g., sample preparation cartridge 114), to which the sample is transferred by components in the analyzer device 200.

In some embodiments, the processing cartridge drawer 230 may receive an AST cartridge (e.g., AST cartridge 115) that is configured to receive aliquots of an enriched sample after processing and enrichment of the sample in the sample preparation cartridge. In additional or alternative embodiments, the processing cartridge drawer 230 may be used as an AST cartridge and/or a PCR cartridge drawer. For example, a PCR cartridge or an AST cartridge may be inserted in the processing cartridge drawer 230 depending on the whether the analyzer device 200 is being used to perform pathogen identification or antimicrobial susceptibility testing of a sample. In some embodiments, the analyzer device 200 may be configured to perform both functionalities of pathogen identification and antimicrobial susceptibility testing with a dual processing cartridge drawer 230 that configured to interface with specialized cartridges or consumables for AST and pathogen identification.

In some embodiments, the sample drawer 210, sample cartridge drawer 220, and processing cartridge drawer 230 may each include a reader configured to scan an identifier of a sample container, sample preparation cartridge, and AST cartridge (or PCR cartridge), respectively. In some embodiments, the readers in the drawers may scan the identifiers of the sample containers and/or cartridges during insertion of each drawer into the housing 201 of the analyzer device 200. In some embodiments, the readers in the drawers 210, 220, and 230 may be configured to scan identification codes, barcodes, or data matrices of corresponding sample containers and/or cartridges. In some embodiments, the readers in the drawers 210, 220, and 230 may be barcode readers, quick response (QR) code readers, or the like.

In some embodiments, FIG. 3A illustrates additional components including one or more centrifuges disposed within the housing 201 and configured to handle tubes and cartridges for sample processing.

FIG. 3B illustrates a diagram of a top view of the analyzer device 200, according to embodiments of the present disclosure. FIG. 3B illustrates a cross-section of analyzer device 200 from a top view, showing the various components, modules, and/or subsystems arranged below the first and second pipettors 202, 204 within the housing 201 of the analyzer device 200.

The housing 201 in FIG. 3B includes the sample drawer 210 containing a plurality of sample containers 212, sample cartridge drawer 220 containing a plurality of sample preparation cartridges 224, and processing cartridge drawer 230 containing both AST cartridges 232 and PCR cartridges 234. Sample containers 212, sample preparation cartridges 224, and AST cartridges 232 represent exemplary embodiments of sample container 111, sample preparation cartridge 114, and AST cartridge 115, shown in FIG. 1, respectively.

In some embodiments, processing cartridge drawer 230 may be configured to hold both AST cartridges 232 and PCR cartridges 234 for performing pathogen identification and antimicrobial susceptibility testing. In some embodiments, there may be a predefined number of sample containers 212, sample preparation cartridges 224, AST cartridges 232, and/or PCR cartridges 234 held in their respective drawers in the housing 201 at a time. In some embodiments, the sample preparation cartridges 224, AST cartridges 232, and/or PCR cartridges 234 may be disposable after a single use or reusable for testing of additional samples.

FIG. 3B also illustrates first centrifuge 240, second centrifuge 242, PCR subsystem 244, enrichment subsystem 246, mechanical apparatus 248, and AST subsystem 250 arranged within housing 201.

In some embodiments, the first centrifuge 240 may be a high-speed centrifuge that is configured to centrifuge processing tubes (e.g., processing tubes 113) and/or spin column baskets that are placed in the first centrifuge 240 by the first pipettor 202. In some embodiments, the second centrifuge 242 may be a low-speed centrifuge that is configured to centrifuge AST cartridges 232 that are placed in the second centrifuge 242 by the second pipettor 204. In some embodiments, the first and second centrifuges 240, 242 may centrifuge processing tubes, spin column baskets, and/or AST cartridges 232 in a swing-bucket configuration. In some embodiments, the first and second centrifuges 240, 242 may comprise a cylindrical shape with a diameter of about 250 mm and a height of about 175 mm.

In some embodiments, the PCR subsystem 244 may comprise a thermal cycler configured to control temperatures when performing quantitative PCR (qPCR) and an optical system for optical interrogation of reaction chambers in a PCR cartridge by fluorescence. In some embodiments, the thermal cycler of the PCR subsystem 244 may control temperatures in a range of about 35° C. to 100° C. In some embodiments, the optical system of the PCR subsystem 244 may perform fluorescent readings from the bottom of a PCR cartridge in the housing 201. In some embodiments, PCR cartridges may be placed in the PCR subsystem 244 and removed from the PCR subsystem 244 by the second pipettor 204. In some embodiments, the PCR subsystem 244 may hold up to two PCR cartridges at a time, in which the PCR cartridges undergo thermal cycling independently in the PCR subsystem 244. In some embodiments, the dimensions of the PCR subsystem 244 may be about 70 mm (width)×125 mm (depth)×250 mm (height).

In some embodiments, enrichment subsystem 246 may be configured to hold a plurality of processing tubes in slots for applying a swinging movement to the processing tubes to allow oscillation of samples and mixing of growth media with pathogens in the samples for growth and enrichment of the pathogens. In some embodiments, the first pipettor 202 may be configured to vertically load tubes into the slots in the enrichment subsystem 246. In some embodiments, the enrichment subsystem 246 may change the orientation of tubes from a vertical position to a horizontal position and apply a swinging movement of ±150 around the horizontal position with a frequency of 1 Hz. In some embodiments, the enrichment subsystem 246 may hold up to about four 15 mL processing tubes at a time, in which each processing tube represents a different sample.

In some embodiments, the enrichment subsystem 246 may be equipped with a moveable magnet. The moveable magnet may engage the processing tubes when in vertical position in the enrichment subsystem 246. In some embodiments, the enrichment subsystem 246 may apply a 37° C. temperature control of processing tubes held in the enrichment subsystem 246. In some embodiments, the dimensions of the enrichment subsystem 246 may be about 150 mm (width)×175 mm (depth)×115 mm (height).

In some embodiments, mechanical apparatus 248 may be configured to agitate a processing tube to perform lysis of microorganisms in samples. In some embodiments, the first pipettor 202 may be configured to vertically load tubes into slots in the mechanical apparatus 248. In some embodiments, the mechanical apparatus 248 may comprise an agitator device or a cell disrupting device that is configured to apply a fast vibration movement to a processing tube. In some embodiments, the mechanical apparatus 248 may apply the fast vibration movement by applying a reciprocating movement along a predetermined axis by the agitator while the processing tube is in a vertical position.

In some embodiments, the mechanical apparatus 248 may hold up to two processing tubes at a time and apply a ±2° angular movement to the processing tubes. In some embodiments, the mechanical apparatus 248 may oscillate processing tubes at about 5,000-30,000 cycles/minute. In some embodiments, the dimensions of the mechanical apparatus 248 may be about 75 mm (width)×175 mm (depth)×100 mm (height).

In additional or alternative embodiments, the mechanical apparatus 248 may comprise a sonicator that is configured to sonicate the processing tube to agitate the sample.

In some embodiments, AST subsystem 250 may comprise a heater configured for incubation of samples in an AST cartridge 232 in the housing 201 and an imaging subsystem for imaging reaction wells of an AST cartridge 232. In some embodiments, the heater of the AST subsystem 250 may be used to control temperatures for incubation of samples in the AST cartridge 232. In some embodiments, the imaging subsystem of the AST subsystem 250 may comprise a microscope configured to acquire images by scanning the bottom of each reaction well of the AST cartridge 232 using a motorized XYZ-translation stage. In some embodiments, the imaging subsystem of the AST subsystem 250 may include a fluorescent sensor, along with two optical channels for detecting different fluorescent signals (e.g., green and red).

In some embodiments, the AST subsystem 250 may identify an antimicrobial phenotypical resistance of the microorganisms (e.g., pathogens) based on the acquired images and/or signals. In some embodiments, AST cartridges 232 may be placed in the AST subsystem 250 and removed from the AST subsystem 250 by the second pipettor 204.

In some embodiments, the AST subsystem 250 may hold up to five AST cartridges 232 at a time. In some embodiments, the AST subsystem 250 may apply a temperature control for AST cartridges 232 held in the AST subsystem 250, such as by using a thermal block. In some embodiments, the AST subsystem 250 may apply about a 37° C. temperature control. In some embodiments, the dimensions of the AST subsystem 250 may be about 200 mm (width)×185 mm (depth)×285 mm (height).

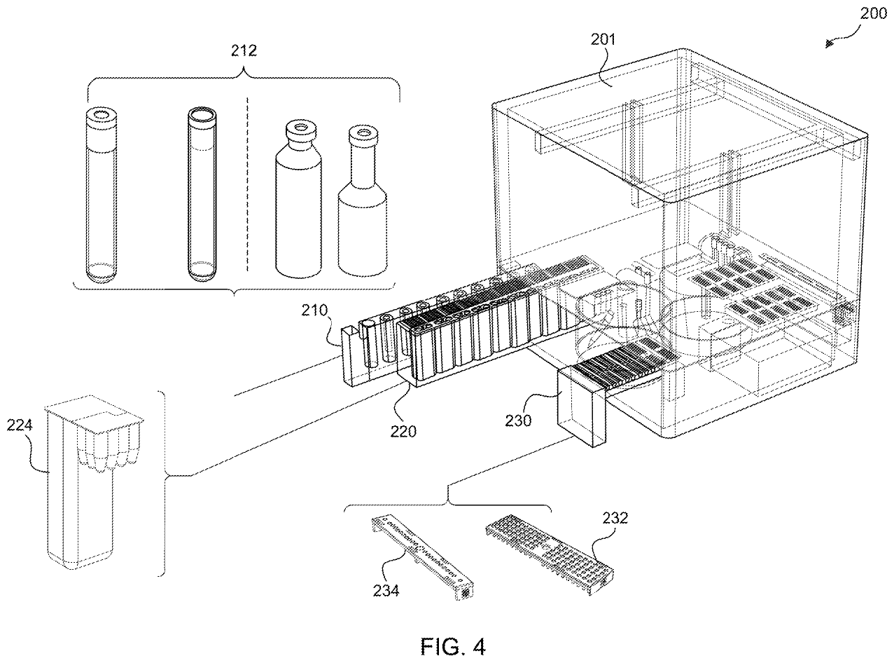

FIG. 4 illustrates a diagram of the analyzer device 200 with three opening compartments, according to embodiments of the present disclosure. In some embodiments, the analyzer device 200 in FIG. 4 shows the sample drawer 210, sample cartridge drawer 220, and processing cartridge drawer 230 extending out from the housing 201 in an open position for loading and/or unloading of samples and/or cartridges. The sample drawer 210, sample cartridge drawer 220, and processing cartridge drawer 230 may be pushed into the housing 201 in a closed position, in which the compartments fit into three corresponding receptacles in the housing 201. In some embodiments, the opening and closing of the sample drawer 210, sample cartridge drawer 220, and the processing cartridge drawer 230 may be controlled by a controller (e.g., controller 109) of the analyzer device 200 and/or by the processing device 116. In some embodiments, a user of the processing device 116 may control the opening and closing of the sample drawer 210, sample cartridge drawer 220, and the processing cartridge drawer 230 by using software installed on the processing device 116. In some embodiments, the sample drawer 210, sample cartridge drawer 220, and the processing cartridge drawer 230 may be manually pulled out and pushed into the housing 201 by a user to load and/or unload sample containers, cartridges, and/or other elements into the analyzer device 200.

In some embodiments, the sample drawer 210 may hold multiple samples. In some embodiments, the sample drawer 210 may hold up to 10 samples at a time, including samples stored in sample containers 212. In some embodiments, the sample containers 212 shown in FIG. 4 may represent one or more blood sample tubes, urine sample tubes, and/or blood culture bottles. In some embodiments, the sample cartridge drawer 220 may hold multiple sample preparation cartridges, such as up to 10 sample preparation cartridges at a time. In some embodiments, the processing cartridge drawer 230 may hold multiple processing cartridges, such as up to 12 PCR cartridges, 6 AST cartridges, or a combination thereof, at a time.

In some embodiments, the dimensions of the sample drawer 210 may be about 40 mm (width)×155 mm (depth)×600 mm (height). In some embodiments, the dimensions of the sample cartridge drawer 220 may be about 85 mm (width)×150 mm (depth)×615 mm (height). In some embodiments, the dimensions of the processing cartridge drawer 230 may be about 140 mm (width)×155 mm (height)×270 mm (depth).

### Embodiments of Sample Processing, Concentration, Enrichment, and Clean-up in Analyzer Device:

In some embodiments, components disposed within the analyzer device 200 may perform sample processing, concentration, enrichment, and clean-up steps in the processing tube 113 before running an AST assay and acquiring images of samples in the AST cartridge 232. In some embodiments, the first pipettor 202 of the analyzer device 200 may transfer a sample from the sample container 111 into the processing tube 113 and perform steps to process, separate, concentrate, and enrich pathogens in the sample for rapid detection.

In some embodiments, sample processing may include a step of blood cell lysis. In embodiments where the sample is a blood sample, the first pipettor 202 may be configured to perform blood cell lysis of the blood sample. The first pipettor 202 may add one or more lysis reagents to the processing tube 113. The one or more lysis reagents may be mixed with the blood sample in the processing tube 113 using a mixer (e.g., such as in enrichment subsystem 246) disposed in the housing 201 of the analyzer device 200 to lyse blood cells in the blood sample. In some embodiments, the one or more lysis reagents may include one or more saponin-based buffers. In some embodiments, the one or more lysis reagents may include one or more detergents, surfactants, or proteases.

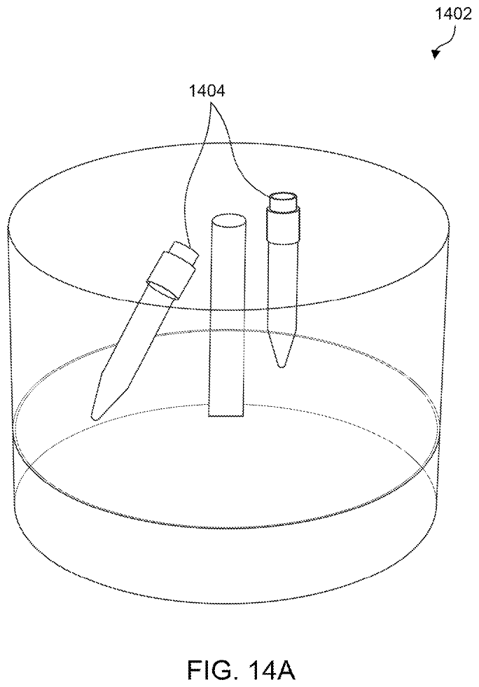

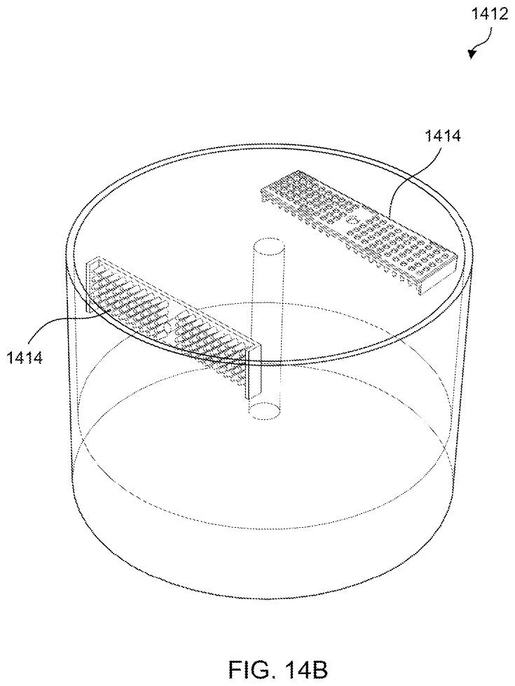

After lysis of the blood cells in the blood sample, the first pipettor 202 may transfer the processing tube 117 to the centrifuge 240 (or 1402 in FIG. 14A) for concentration and enrichment of the lysed sample. Concentration and enrichment of the sample may involve a series of steps using components in the analyzer device 200, including centrifugation. In some embodiments, the first pipettor 202 in the analyzer device 200 may move the processing tube 117 to a centrifuge, such as first centrifuge 240 or 1402. In some embodiments, the centrifuge 240 or 1402 may apply centrifugal force to the processing tube 117 to concentrate the pathogens in the sample, such as by separating the pathogens from other components in the sample.

After centrifuging, the first pipettor 202 may be used to remove a fluid from the processing tube 117, leaving the concentrated pathogens in the processing tube 117. In some embodiments, the first pipettor 202 may then add a growth media to the concentrated pathogens in the processing tube 117 to grow the concentrated pathogens in the processing tube 117 for a predetermined period of time. In some embodiments, the predetermined period of time may be a period of time that allows growth of the concentrated pathogens, such as about 4 hours or more. In some embodiments, the predetermined period of time may be 3-4 hours for the enrichment of most pathogens, whereas other pathogens may take longer time periods for growth/enrichment. In some embodiments, the growth media may be stored in one or more reservoirs of the sample preparation cartridge 224. In some embodiments, the growth media may comprise Mueller Hinton broth, cation-adjusted Mueller Hinton broth, Tryptic Soy broth, Lysogeny broth, Brain Heart Infusion (BHI) broth, or the like.

After concentration and enrichment, the analyzer device 200 may perform a cleaning or clean-up step of the sample to obtain the pathogens in the enriched sample. In some embodiments, in order to clean the concentrated pathogens, the first pipettor 202 may move the processing tube 117 to the centrifuge 240 or 1402 after the predetermined period of time for growth has elapsed (after adding the growth media) and remove a supernatant from the processing tube 117 to obtain the enriched sample.

In some embodiments, the analyzer device 200 may implement the clean-up step of the sample by immunomagnetic separation (IMS) techniques, such as by using magnetic beads. In some embodiments, the processing tube 117 may include a plurality of magnetic beads configured to attach to the concentrated pathogens in the processing tube 117. In some embodiments, the magnetic beads may be coated with non-specific ligands. In some embodiments, the magnetic beads are coated with specific ligands that are specific to a particular pathogen or groups of pathogens of the concentrated pathogens in the processing tube 117. In some embodiments, the pathogens may be immobilized on the surface of the magnetic beads after an incubation period and can be concentrated into a pellet using a magnetic field.

In some embodiments, the first pipettor 202 may move the processing tube 117 to a magnet station, such as in enrichment subsystem 246, to apply a magnetic force to the processing tube 117 to retain the concentrated pathogens attached to the magnetic beads. In some embodiments, the first pipettor 202 may then remove extraneous liquid from the processing tube 117 after retention of the concentrated pathogens attached to the magnetic beads in the processing tube 117. The removal of the extraneous liquid may result in the enriched sample with the concentrated pathogens.

In some embodiments, the concentrated pathogens may be re-suspended and washed to remove any debris or other materials in a wash step. In particular, the first pipettor 202 may add one or more wash materials to the concentrated pathogens in the processing tube 117 to clean and remove any blood components or debris from the concentrated pathogens, leaving the enriched sample in the processing tube 117. In some embodiments, the one or more wash materials may include a combination of one or more buffers, detergents, surfactants, and proteases. In some embodiments, the one or more wash materials may be stored in the sample preparation cartridge 224.

In some embodiments, the analyzer 200 may be configured to identify a number of pathogens in a sample. In particular, the analyzer 200 may use one or more fluorescent dyes and a microscope (e.g., in the AST subsystem 250) for labeling cells in the sample and counting the number of pathogens. In some embodiments, the number of pathogens in an initial sample (e.g., prior to concentration and enrichment) may be less than 200. By performing concentration and enrichment steps, the number of the pathogens in the enriched sample may be increased to a range of about 1,000 to about 100,000. In some embodiments, the number of the pathogens in the enriched sample may be about 10,000. In response to the identification of the number of pathogens in the sample, the analyzer 200 may be configured to further enrich or dilute the sample to obtain a predetermined number of pathogens in the enriched sample.

In some embodiments, the sample may be treated with protease and/or DNAse before or after concentration. For example, protease and/or DNAse may be added to the sample after concentration but before enrichment. In another example, protease and/or DNAse may be added to the sample at the same time as lysis reagents.

In some embodiments, the sample may be treated with protease and/or DNAse during the clean-up process. For example, proteases and/or DNAse may be added during the immunomagnetic capture process to digest and reduce the amount of debris in the sample. In another example, proteases and/or DNAse may be added after the immunomagnetic capture process, which has the advantage of avoiding any interference of proteases/DNAses reagents into the IMS process. This is of particular importance when the capture of the bacteria by the beads is based in proteins which may be degraded by the proteases and hinder their effect.

After concentration, enrichment, and clean-up, the enriched sample in the processing tube 117 may subsequently be ready for transfer to the AST cartridge 232 by the pipetting system in the analyzer 200 for performing the AST assay and image acquisition for determining susceptibility of the concentrated pathogens in the enriched sample.

### Sample Preparation Cartridge Embodiments:

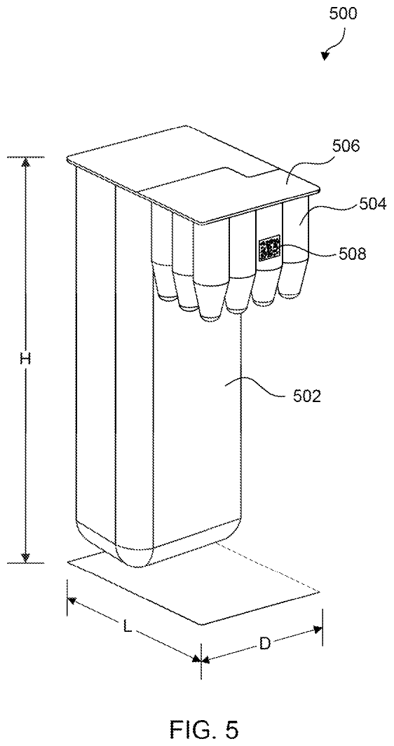

FIG. 5 illustrates a diagram of a sample preparation cartridge 500, according to embodiments of the present disclosure. Sample preparation cartridge 500 represents an exemplary embodiment of sample preparation cartridge 224 shown in FIG. 3B. The sample preparation cartridge 500 may be a consumable that is inserted into the sample drawer 210 of the analyzer device 200 for preparing and processing a sample in a sample container. In some embodiments, the sample preparation cartridge 500 may be made by injection molding from a polypropylene (PP) material. The sample preparation cartridge 500 may comprise a housing 502, a plurality of reservoirs 504, a lid 506, and an identifier 508.

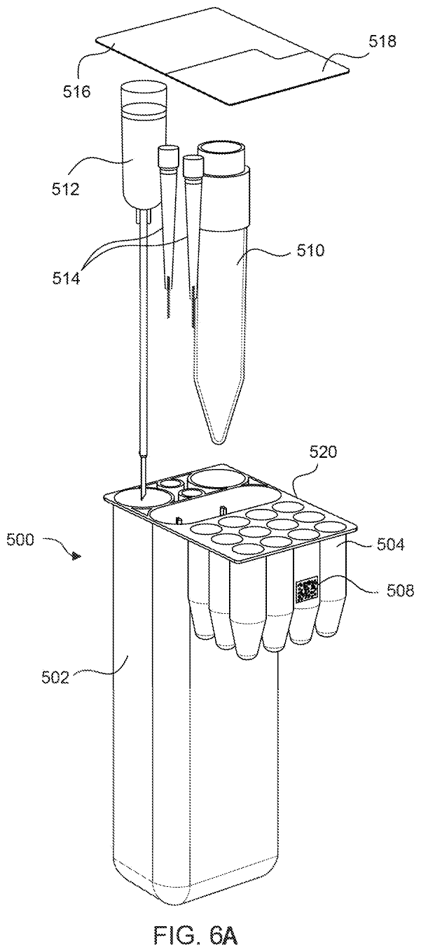

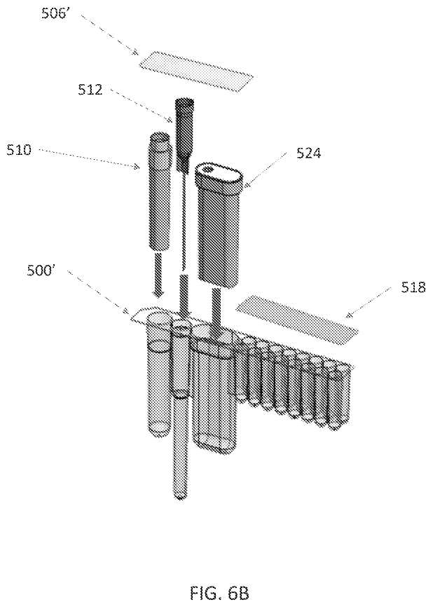

In some embodiments, the housing 502 may have an elongated rectangular shape with rounded edges. The housing 502 may be configured to hold additional elements used for sample preparation as shown in FIG. 6A. In some embodiments, the plurality of reservoirs 504 may be separate reservoirs or tubes molded together. The plurality of reservoirs 504 may be configured to store materials used for performing sample concentration, lysis, and/or nucleic acid amplification. In some embodiments, materials stored in the reservoirs 504 may include one or more buffers (e.g., NaCl-based buffers, phosphate-buffered saline (PBS, or the like), detergents, surfactants, proteases, growth media, or the like. In some embodiments, the reservoirs 504 may store one or more lysis reagents, such as one or more saponin-based buffers, detergents, surfactants, or proteases, for performing cell lysis of a blood sample. In some embodiments, the reservoirs 504 may store one or more wash materials, which may include a combination of one or more buffers, detergents, surfactants, and proteases.

In some embodiments, the lid 506 of the sample preparation cartridge 500 is a protective lid that extends across and covers the housing 502 and the plurality of reservoirs 504. In some embodiments, the identifier 508 is an identifier of the sample preparation cartridge 500 that may scanned by the analyzer device 200 for performing sample preparation for antimicrobial susceptibility testing. In some embodiments, the identifier 508 may be at least one of an identification code, barcode, or data matrix, such as a QR code. In some embodiments, the dimensions of the sample preparation cartridge 500 may be about 80 mm (width)×55 mm (depth)×155 mm (height) as shown in FIG. 5.

FIG. 6A illustrates an exploded-view diagram of the sample preparation cartridge 500 with a processing tube 510 and other components to be inserted therein, according to embodiments of the present disclosure. The sample preparation cartridge 500 may include processing tube 510, a first removable needle 512, and two second removable needles 514 that are stored in corresponding receptacles in the housing 502 of the sample preparation cartridge 500. Processing tube 510 represents an exemplary embodiment of processing tube 113 shown in FIG. 1.

In some embodiments, the processing tube 510 may comprise, for example, a 15 ml tube with a conical bottom. In some embodiments, the processing tube 510 may be removable from the sample preparation cartridge 500 for processing in other modules in the analyzer device 200. In some embodiments, the processing tube 510 may be the tube to which a sample is transferred after loading of a sample container and the sample preparation cartridge 500 into the sample drawer 210 and sample cartridge drawer 220, respectively, of the analyzer device 200.

In some embodiments, the sample may be transferred by the first removable needle 512 from a sample container in the sample drawer 210 to the processing tube 510 of the sample preparation cartridge 500 in the sample cartridge drawer 220. In some embodiments, the first pipettor 202 may attach to the first removable needle 512, which is configured to transfer the sample to and/or from the processing tube 510 by insertion of the first removable needle 512 through a septum of the processing tube 510. In some embodiments, the two second removable needles 514 may be used to handle low volumes, and the second pipettor 204 may be configured to attach to the two second removable needles 514.

FIG. 6A also illustrates the openings 520 of the plurality of reservoirs 504 of the sample preparation cartridge 500. In some embodiments, there may be 12 reservoirs 504 in the sample preparation cartridge 500, in which each reservoir 504 holds a volume of about 2.5 mL. In some embodiments, the sample preparation cartridge 500 may include a pierceable film 516 that covers the receptacles of the housing 502, and/or a foil seal 518 that covers the openings 520 of the plurality of reservoirs 504. In some embodiments, the pierceable film 516 may be a polyester film, and the foil seal 518 may comprise an aluminum foil, in which both the pierceable film 516 and the foil seal 518 are pierceable by the first and/or second removable needles 512, 514.

FIG. 6B illustrates an exploded-view diagram of a sample preparation cartridge 500′ with a processing tube 510 and other components to be inserted therein, according to embodiments of the present disclosure. Sample preparation cartridge 500′ is similar to sample preparation cartridge 500, but with a more linear form factor and a peelable film 506′ instead of lid 506.

### Processing Tube and Needle Embodiments:

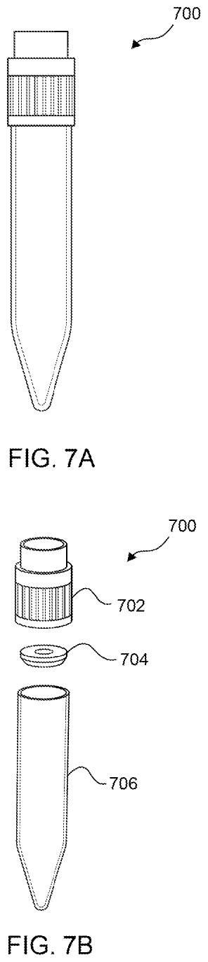

FIGS. 7A and 7B illustrate diagrams of a processing tube 700, according to embodiments of the present disclosure. Processing tube 700 represents an exemplary embodiment of processing tube 510 shown in FIG. 6A. In particular, FIG. 7A illustrates the processing tube 700 after assembly, whereas FIG. 7B illustrates an exploded view of the components in the processing tube 700. The processing tube 700 comprises a cap 702, a septum 704, and a tube 706.

In some embodiments, the septum 704 may be affixed inside the tube 706 with the cap 702 fitted over the septum 704 of the processing tube 700. In some embodiments, the septum 704 may be configured for insertion and removal of needles (e.g., needles 512 and 514) for transfer of liquids to and from the processing tube 700 without necessitating removal of the cap 702 from the tube 706. In some embodiments, the septum 704 may provide an airtight seal within the processing tube 700 and prevent contamination of the contents of the processing tube 700.

In some embodiments, the processing tube 700 may comprise a handling feature at a top end of the processing tube 700 that is compatible for handling by a pipettor (e.g., first and/or second pipettors 202, 204). In some embodiments, the handling feature of the processing tube 700 may be a cylindrical cavity in the cap 702 that is compatible for insertion by a tip of a pipettor. In some embodiments, the pipette tip of the first and second pipettors 202, 204 may press and fit into the cylindrical cavity in the cap 702 to pick up and move the processing tube around in the analyzer device 200.

In some embodiments, the cap 702 may be made of a high-density polyethylene (HDPE) material, and the tube 706 may be made from a polypropylene (PP) material. In some embodiments, the septum 704 may comprise at least one of a rubber, polytetrafluoroethylene (PTFE), thermoplastic elastomer (TPE), silicone, butyl rubber, or a combination thereof. In some embodiments, the septum 704 may comprise a double layer of polytetrafluoroethylene (PTFE) and another material selected from the group consisting of silicone, rubber, and butyl rubber. In some embodiments, the dimensions of the processing tube 700 after assembly may comprise a height of, for example, about 110 mm and a diameter of about 20 mm. In some embodiments, the septum 704 may have a thickness in a range of about 1 to 2 mm.

In some embodiments, the processing tube 700 may include a plurality of magnetic beads configured to attach to concentrated pathogens in the processing tube 700. In some embodiments, the plurality of magnetic beads may be coated with non-specific ligands. In some embodiments, the plurality of magnetic beads may be coated with specific ligands that are specific to a particular pathogen of the concentrated pathogens in the processing tube 700. In additional or alternative embodiments, the plurality of magnetic beads may be stored in one or more reservoirs 504 of the sample preparation cartridge 500. In some embodiments, the processing tube 700 may include a plurality of beads configured to lyse concentrated pathogens in the processing tube 700.

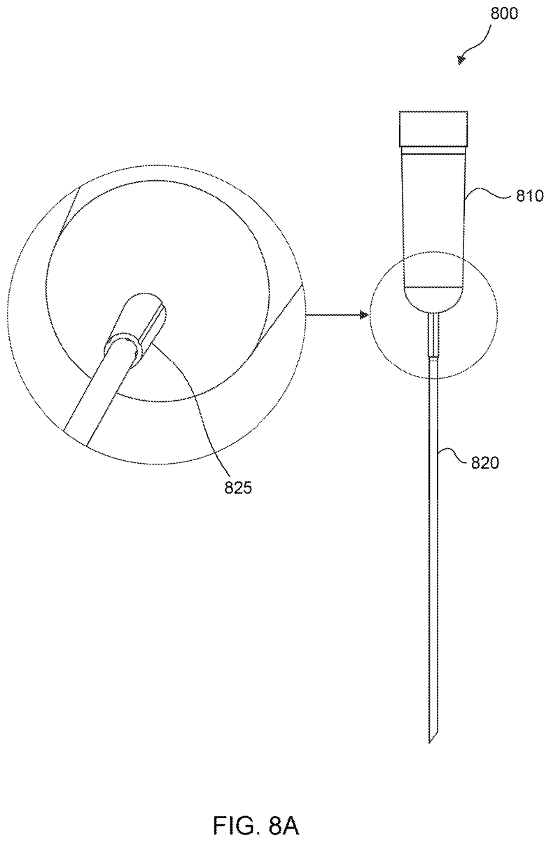

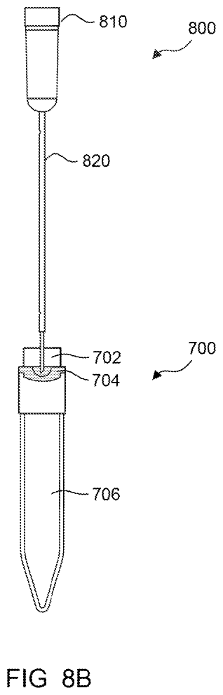

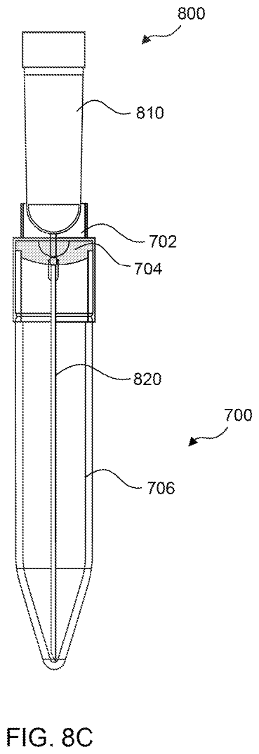

In some embodiments, the processing tube 700 may be configured to receive needles as shown in FIGS. 8A, 8B, and 8C. FIGS. 8A, 8B, and 8C illustrate diagrams of a needle 800 configured for insertion into a processing tube 700, according to some embodiments of the present disclosure.

In particular, FIG. 8A illustrates needle 800 comprising a plastic body 810, a cannula 820, and a plurality of slots 825. In some embodiments, the plastic body 810 may be attached to the cannula 820 by bonding. In some embodiments, the pipettor(s) in the analyzer device (e.g., first pipettor 202) may attach to the proximal end of the plastic body 810 of the needle 800. In some embodiments, the plastic body 810 of the needle 800 includes an aerosol filter configured to prevent contamination of the pipettors in the analyzer device. In some embodiments, needle 800 may be a venting needle that is configured to vent the processing tube 700 upon insertion of the needle. In some embodiments, venting of the needle 800 may facilitate in relieving pressure in a sealed processing tube 700. In some embodiments, the plastic body 810 of the needle 800 comprises a predetermined number of slots 825 configured to provide an air connection between an inside and an outside of the processing tube 700 when the needle 800 is inserted through the septum 704 and into the tube 706. For example, there may be four slots 825 in the injected part of the needle 800 that holds the cannula 820. In some embodiments, the slots 825 may be generated by injection molding.

FIG. 8B illustrates the needle 800 during insertion into the processing tube 700, and FIG. 8C illustrates the needle 800 after full insertion into the processing tube 700. In some embodiments, the cannula 820 of the needle 800 may be inserted into the cap 702, through the septum 704, and into the tube 706 of the processing tube 700. In some embodiments, the distal end of the plastic body 810 may fit into the cap 702 of the processing tube 700 upon full insertion of the cannula 820 into the tube 706.

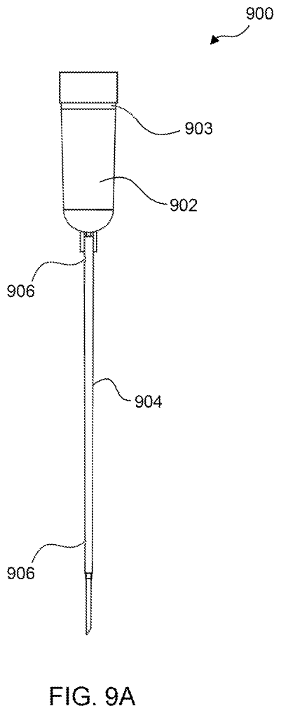

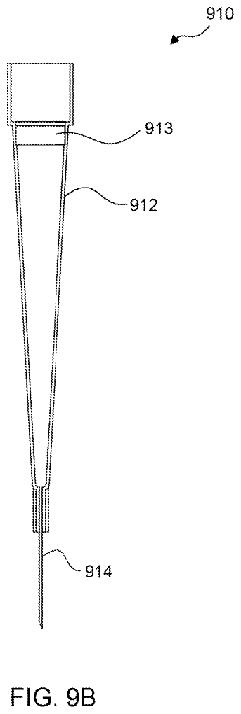

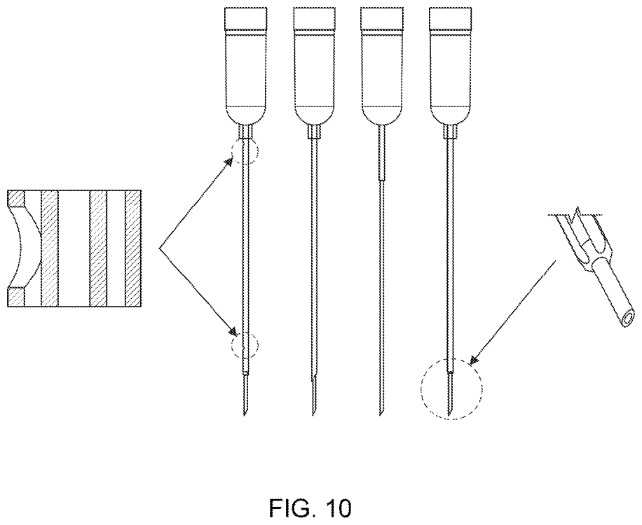

FIGS. 9A and 9B illustrate diagrams of a high volume needle 900 and a low volume needle 910, respectively, according to embodiments of the present disclosure. In some embodiments, high volume needle 900 and low volume needle 910 may be coupled to first pipettor 202 and second pipettor 204, respectively, in the analyzer device 200.

As shown in FIG. 9A, the high volume needle 900 may comprise a plastic body 902 and a cannula 904. In some embodiments, the plastic body 902 may be a reservoir configured to hold a volume of about 50 μL to 5 mL during liquid transfer in the analyzer device. In some embodiments, the plastic body 902 may include a filter 903 arranged within to prevent contamination of a pipettor coupled to the high volume needle 900.

In some embodiments, the plastic body 902 may be made of a polypropylene (PP) material. In some embodiments, the plastic body 902 of the needle 900 may have a diameter of about 16 mm and a length of about 50 mm. In some embodiments, the cannula 904 may be made of stainless steel. In some embodiments, the cannula 904 may have a length of about 100 mm. In some embodiments, the plastic body 902 and cannula 904 when assembled together may have a length of about 150 mm. In some embodiments, the high volume needle 900 may be a 17 gauge needle with an inner diameter (ID) of about 1.05 mm, an outer diameter (OD) of about 1.60 mm, and a cannula diameter (CD) of about 2.50 mm.

In some embodiments, the cannula 904 may further comprise a secondary cannula arranged around an inner core of the needle 900. In some embodiments, the cannula 904 comprises one or more venting holes 906. In some embodiments, the cannula 904 of the needle 900 may be in fluid communication with the venting hole 906. In some embodiments, the cannula 904 may be a slotted cannula with slots around the needle.

As shown in FIG. 9B, the low volume needle 910 may comprise a plastic body 912 and a needle shaft 914. In some embodiments, the plastic body 912 may be a reservoir configured to hold a volume of about 1 to 200 μL during liquid transfer in the analyzer device. In some embodiments, the plastic body 912 may include a filter 913 arranged within to prevent contamination of a pipettor coupled to the low volume needle 910. In some embodiments, the plastic body 912 may be made of a polypropylene (PP) material. In some embodiments, the plastic body 912 of the needle 910 may have a diameter of about 7.25 mm and a length of about 45 mm. In some embodiments, the needle shaft 914 may have a length of about 10 mm. In some embodiments, the needle shaft 914 may be made of stainless steel. In some embodiments, the low volume needle 910 may be a 29 gauge needle with an inner diameter (ID) of about 0.20 mm and an outer diameter (OD) of about 0.30 mm.

FIG. 10 illustrates a diagram of examples of the high volume needle 900, according to embodiments of the present disclosure. In particular, FIG. 10 illustrates various examples of the cannula of the high volume needle 900 with venting holes, slots, and the like. In some embodiments, the first needle shown in FIG. 10 may vent by having two connected orifices or venting holes in the cannula of the needle. In some embodiments, the second and fourth needles shown in FIG. 10 may vent when the tip of each needle is inserted into the septum (e.g. septum 704) of the processing tube.

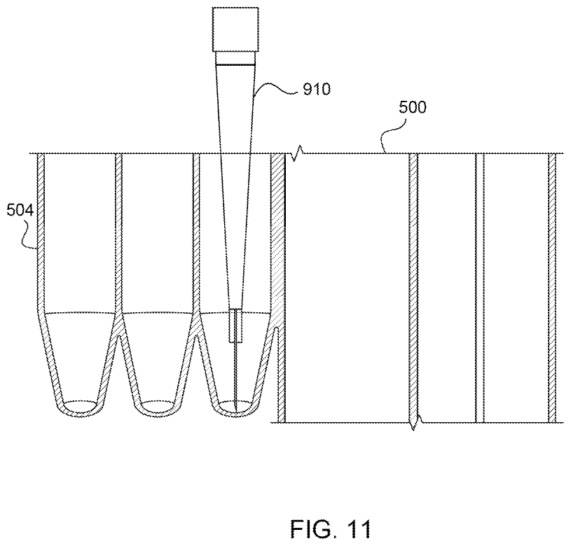

FIG. 11 illustrates a diagram of the low volume needle 910 interfacing with the sample preparation cartridge 500, according to embodiments of the present disclosure. In particular, FIG. 11 shows an example of the low volume needle 910 disposed in one of the plurality of reservoirs 504 of the sample preparation cartridge 500, such as for transferring materials used for sample concentration and/or lysis from the reservoirs 504 to a sample in the processing tube. In some embodiments, the sample preparation cartridge 500 may be configured to receive one needle, such as low volume needle 910. In other embodiments, the sample preparation cartridge 500 may be configured to receive two needles, such as both high volume needle 900 and low volume needle 910.

### AST Cartridge Embodiments:

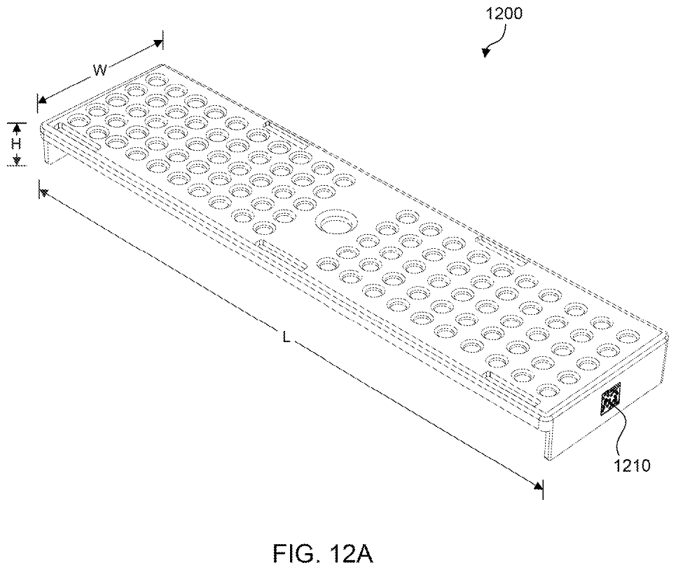

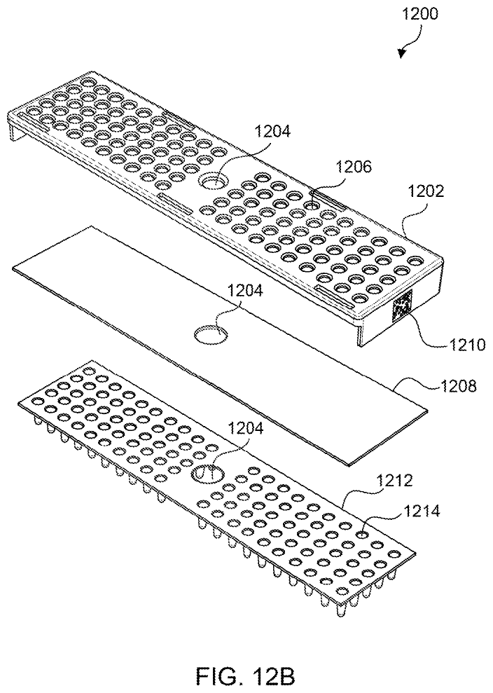

FIGS. 12A and 12B illustrate diagrams of an AST cartridge 1200, according to embodiments of the present disclosure. AST cartridge 1200 represents an exemplary embodiment of AST cartridge 232. In particular, FIG. 12A illustrates the AST cartridge 1200 after assembly, whereas FIG. 12B illustrates an exploded view of the components in the AST cartridge 1200. The AST cartridge 1200 comprises a cover 1202, a septum 1208, and a base 1212. In some embodiments, the cover 1202 is arranged over the septum 1208, and the septum is arranged over the base 1212.

In some embodiments, the base 1212 comprises a plurality of reaction wells 1214. In some embodiments, the number of reaction wells 1214 in the base 1212 may be in a range of about 50 to 200, for example, 100 reaction wells. In some embodiments, each reaction well 1214 may hold a volume in a range of about 20 to 50 μL, for example, 30 μL. Each reaction well 1214 in the plurality of reaction wells 1214 may contain an antimicrobial of a predetermined concentration for reacting with a respective aliquot of an enriched sample comprising pathogens. In some embodiments, the antimicrobial disposed within each reaction well 1214 may be in a liquid form, or in a dried or freeze dried form. In some embodiments, the antimicrobial disposed within each reaction well 1214 may be added to each reaction well 1214 in the base 1212 by the first or second pipettor 202, 204 prior to the dispensing the plurality of aliquots of the enriched sample to the reaction wells 1214. In some embodiments, the antimicrobial disposed within each reaction well 1214 may be added to each reaction well 1214 during manufacturing and/or assembly of the AST cartridge 1200.

In some embodiments, the first or second pipettor 202, 204 in the analyzer device 200 may dispense a plurality of aliquots of an enriched sample to the reaction wells 1214 in the AST cartridge 1200 after concentration and enrichment of pathogens in a sample in the processing tube 700. In some embodiments, each reaction well 1214 may be configured to receive the aliquot of the enriched sample comprising pathogens by a contactless dispensing of the aliquot by a needle (e.g., needle 900 or 910 coupled to the first or second pipettor 202, 204). In some embodiments, the needle may penetrate the septum 1208 of each reaction well 1214 to dispense each aliquot without the needle contacting the bottom surface of each reaction well 1214. In some embodiments, a volume of each aliquot dispensed by the needle 900 or 910 may be in a range of about 0.5 μL to about 10 μL.

In some embodiments, each reaction well 1214 comprises a conical shape and a bottom wall. In some embodiments, the bottom wall of each reaction well 1214 may have a diameter that is less than about 2 mm. In some embodiments, the contactless dispensing of the plurality of aliquots to each reaction well 1214 may include using jet dispensing to perform a contactless dispensing of the aliquots by needle 900 or 910 in the sample preparation cartridge 500 penetrating respective septums of the reaction wells 1214 and without the needle 900 or 910 contacting a bottom wall of each reaction well 1214.

In some embodiments, the bottom wall of each reaction well 1214 may be optically transparent and configured for optical interrogation. In some embodiments, the bottom wall of each reaction well 1214 may be configured for optical interrogation, such as by AST subsystem 250 in analyzer device 200. In some embodiments, the bottom wall of each reaction well 1214 may be configured for fluorescence microscopy, such as by AST subsystem 250 in analyzer device 200. In some embodiments, the plurality of reaction wells 1214 may be configured to fit into corresponding wells in a temperature control block in the AST subsystem 250, and the temperature control block may be configured to heat the sides of the plurality of reaction wells 1214.

The septum 1208 seals each reaction well 1214 in the plurality of reaction wells 1214 of the base 1212. In some embodiments, the septum 1208 may be referred to as a sealing cap mat. In some embodiments, the septum 1208 may comprise a unibody that extends across the plurality of reaction wells 1214 of the base 1212. In some embodiment, the septum 1208 may comprise multiple parts assembled together, wherein each part covers each reaction well 1214 of the base 1212. The septum 1208 may be configured to receive a needle, such as needle 900 or 910 coupled to the first or second pipettor 202, 204. In some embodiments, the needle may create orifices in the septum 1208 during insertion, and the orifices in the septum 1208 may close up when the needle is removed as a result of the material of the septum 1208. In some embodiments, the septum 1208 may comprise at least one of a rubber, polytetrafluoroethylene (PTFE), thermoplastic elastomer (TPE), silicone, butyl rubber, or a combination thereof. In some embodiments, the septum 1208 may comprise a double layer of polytetrafluoroethylene (PTFE) and another material selected from the group consisting of silicone, rubber, and butyl rubber. In some embodiments, the septum 1208 may have a thickness in a range of about 1 to 2 mm.

In some embodiments, the cover 1202 may comprise a plurality of openings 1206, wherein each opening 1206 aligns with a respective reaction well 1214 of the plurality of reaction wells 1214 in the base 1212. In some embodiments, the cover 1202 may also include an identifier 1210. The identifier 1210 may be an identifier of the AST cartridge 1200 that may be scanned by the analyzer device 200 for performing antimicrobial susceptibility testing. In some embodiments, the identifier 1210 may be at least one of an identification code, barcode, or data matrix.

In some embodiments, the cover 1202 may fit over the septum 1208 and base 1212 to form an assembled AST cartridge 1200. In some embodiments, the septum 1208 may be overmolded into the cover 1202 to form a combined component, and the combined component may be assembled over the base 1212 by at least one of a snap-fit joint or a mechanical fastener. In some embodiments, the cover 1202, septum 1208, and base 1212 may be interlocked or clamped together by one or more mechanical fasteners.

In some embodiments, the base 1212, the septum 1208, and the cover 1202 each comprise an opening 1204 in a center of the AST cartridge 1200. In some embodiments, the opening 1204 may be a circular hole that is compatible for insertion by a pipette tip (e.g., first and second pipettors 202, 204) for moving the AST cartridge 1200 in the analyzer device 200. In some embodiments, the opening 1204 in the base 1212, the septum 1208, and the cover 1202 may align with each other upon assembly of the AST cartridge 1200.

In some embodiments, the base 1212 may be made of a polystyrene (PS) material. In some embodiments, the cover 1202 may be made of a polypropylene (PP) or a polycarbonate (PC) material. In some embodiments, the dimensions of the assembled AST cartridge 1200 may be about 135 mm (length)×35 mm (width)×10 mm (height).

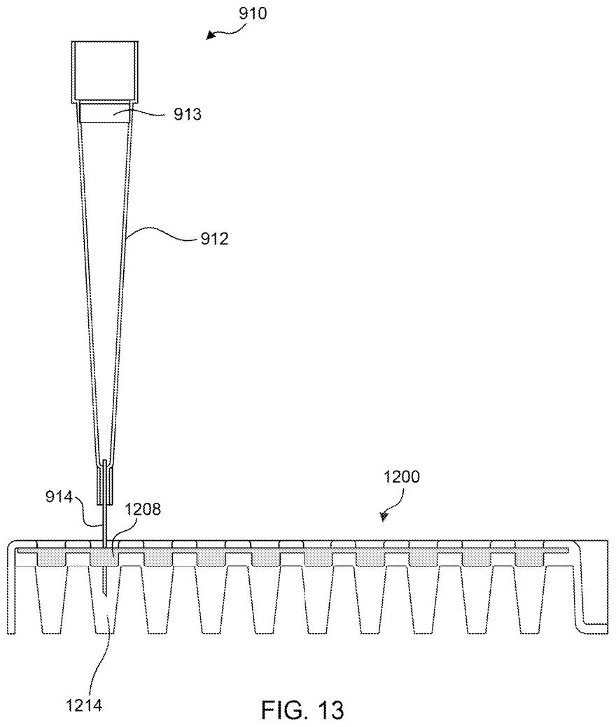

FIG. 13 illustrates a diagram of the low volume needle 910 being inserted into the AST cartridge 1200, according to embodiments of the present disclosure. In particular, FIG. 13 shows the needle shaft 914 of the needle 910 piercing through the septum 1208 and into the reaction well 1214 of the AST cartridge 1200. In some embodiments, the needle shaft 914 may create orifices in the septum 1208 during insertion. The orifices in the septum 1208 may close up when the needle shaft 914 is removed as a result of the material of the septum 1208.

### Embodiments of Modules and Subsystems in the Analyzer:

FIGS. 14A and 14B illustrate diagrams of example centrifuges used in the analyzer device 200, according to embodiments of the present disclosure. In particular, FIG. 14A illustrates a first centrifuge 1402 that may be used to centrifuge samples in a processing tube 1404, whereas FIG. 14B illustrates a second centrifuge 1412 that can be used to centrifuge enriched samples in an AST cartridge 1414. First and second centrifuges 1402, 1412 represent exemplary embodiments of first and second centrifuges 240, 242 shown in FIG. 3B, respectively. Processing tube 1404 and AST cartridge 1414 represent exemplary embodiments of processing tube 700 and AST cartridge 1200, shown in FIGS. 7A-7B and FIGS. 12A-12B, respectively.

In some embodiments, the first centrifuge 1402 may be a high-speed centrifuge that is configured to apply a relative centrifugal force (RCF) or g force of about 12,000 G to the processing tube 1404. In some embodiments, the second centrifuge 1412 may be a low-speed centrifuge that is configured to apply a relative centrifugal force (RCF) or g force of about 3,000 G AST cartridge 1414.

In some embodiments, the first centrifuge 1402 may hold the processing tube 1404 in a first orientation and apply a 450 swing-bucket centrifugation to the processing tube 1404. In some embodiments, the second centrifuge 1412 may hold the AST cartridge 1414 in another orientation and apply a 900 swing-bucket centrifugation, such that the AST cartridge 1414 moves to a vertical position in the second centrifuge 1412.

In some embodiments, the first centrifuge 1402 and the second centrifuge 1412 may centrifuge multiple processing tubes 1404 and AST cartridges 1414, respectively, at a time. For example, the first centrifuge 1402 may be configured to hold two processing tubes 1404 at a time for centrifuging together. In another example, the second centrifuge 1412 may be configured to hold two AST cartridges 1414 at a time for centrifuging together. In some embodiments, processing tubes 1404 may be moved into the first centrifuge 1402 during the concentration and enrichment steps for obtaining an enriched sample with pathogens.

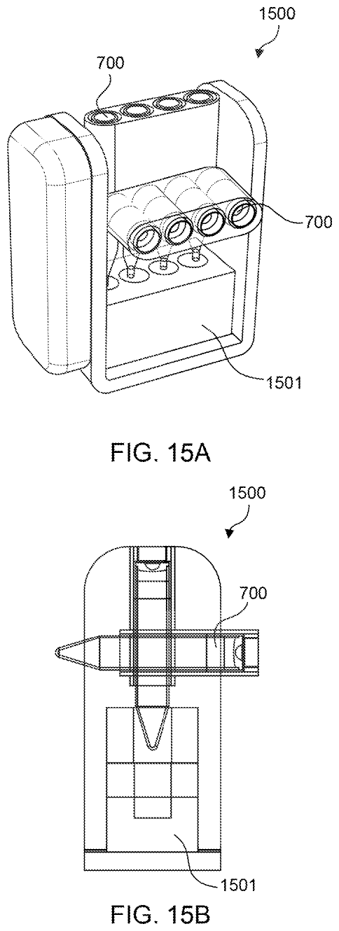

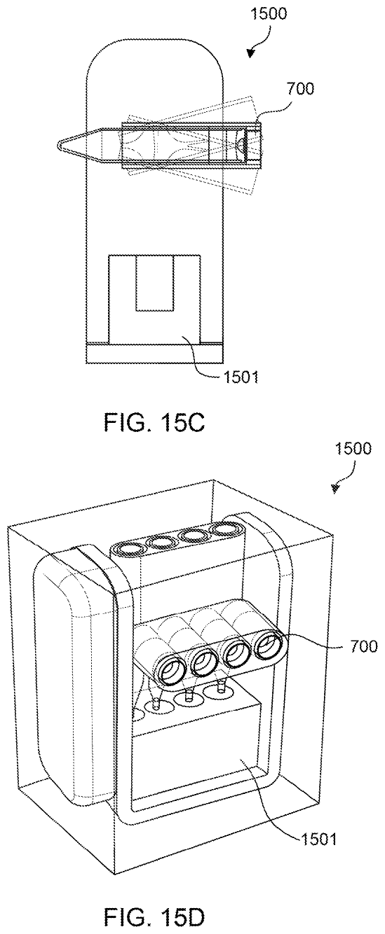

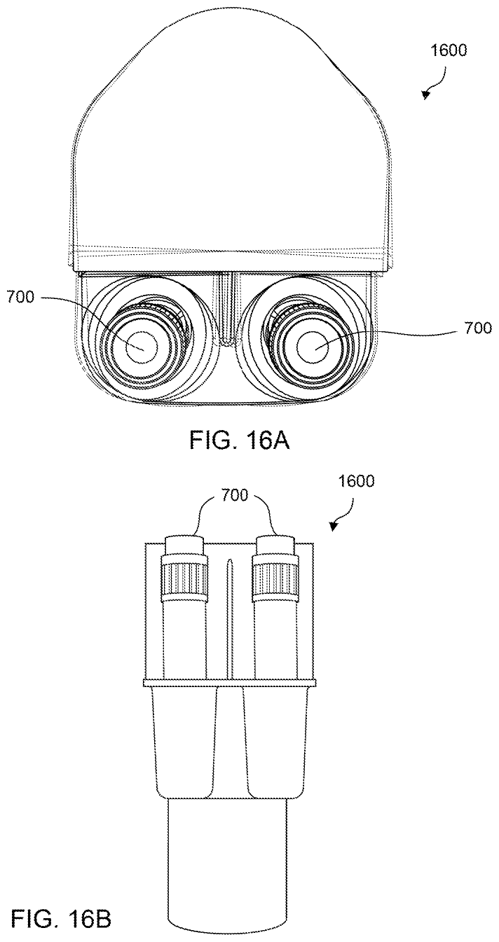

FIGS. 15A, 15B, 15C, and 15D illustrate diagrams of an example enrichment subsystem 1500 used in the analyzer device 200, according to embodiments of the present disclosure. Enrichment subsystem 1500 represents an exemplary embodiment of enrichment subsystem 246 shown in FIG. 3B. In some embodiments, enrichment subsystem 1500 may apply a swinging motion to processing tubes 700 to allow oscillation and mixing of the samples with other materials. In some embodiments, the processing tube 700 may be placed in the enrichment subsystem 1500 by the pipetting system (e.g., first or second pipettor 202, 204) for mixing of the pathogens in processing tube 700 with growth media, for pathogen growth and enrichment of the sample. In some embodiments, the processing tube 700 may be placed in the enrichment subsystem 1500 by the pipetting system (e.g., first or second pipettor 202, 204) for mixing one or more lysis reagents with a blood sample in the processing tube 700 to lyse blood cells in the blood sample. In some embodiments, the mixing functionality of the enrichment subsystem 1500 may be used for sample processing for both pathogen identification and/or antimicrobial susceptibility testing in the analyzer device 200.

In some embodiments, the enrichment subsystem 1500 may be a mixer, such that it rotates one or more processing tubes 700 in a horizontal position by swinging the processing tubes 700 back and forth at a ±30° angle. In some embodiments, four processing tubes 700 may be loaded into the enrichment subsystem 1500 at a time. In some embodiments, the enrichment subsystem 1500 may include a magnet 1501 that moves back and forth between an up position and a down position. In some embodiments, one or more processing tubes 700 may include magnetic beads that are configured to attach to concentrated pathogens in the one or more processing tubes 700. In some embodiments, the magnet 1501 may be used to retain the concentrated pathogens attached to the magnetic beads in the one or more processing tubes 700.

In some embodiments, the enrichment subsystem 1500 may include an additional or alternative independent magnet station that may be used to retain concentrated pathogens attached to magnetic beads in the processing tube 700 and/or in the AST cartridge 1200. In some embodiments, the magnet station in the enrichment subsystem 1500 may be used to move pathogens in each aliquot of an enriched sample (e.g., in the reaction wells 1214 of the AST cartridge 1200) to a bottom wall of each reaction well 1214 for acquiring the image of the AST cartridge 1200. In some embodiments, the pathogens in each aliquot may be attached to magnetic beads that allow movement of the pathogens to the bottom wall of the reaction wells 1214.

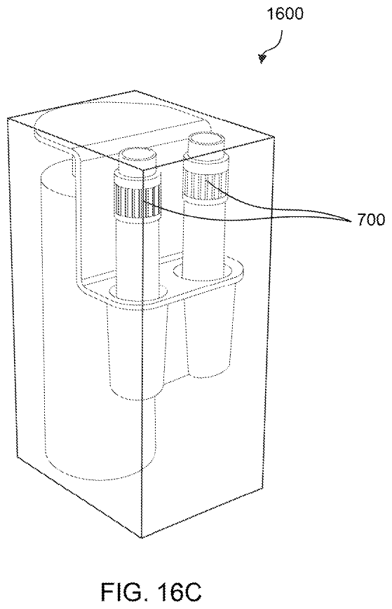

FIGS. 16A, 16B, and 16C illustrate diagrams of an example mechanical apparatus 1600 used in the analyzer device 200, according to embodiments of the present disclosure. Mechanical apparatus 1500 represents an exemplary embodiment of mechanical apparatus 248 shown in FIG. 3B. In some embodiments, mechanical apparatus 1600 may hold two processing tubes 700 in a vertical position and provide fast vibrational movements to the processing tubes 700, such as for performing lysis of microorganisms in samples. In some embodiments, mechanical apparatus 1600 may be an agitator. In additional or alternative embodiments, the mechanical apparatus 1600 may include a sonicator that is configured to sonicate the processing tube 700 to agitate the sample.

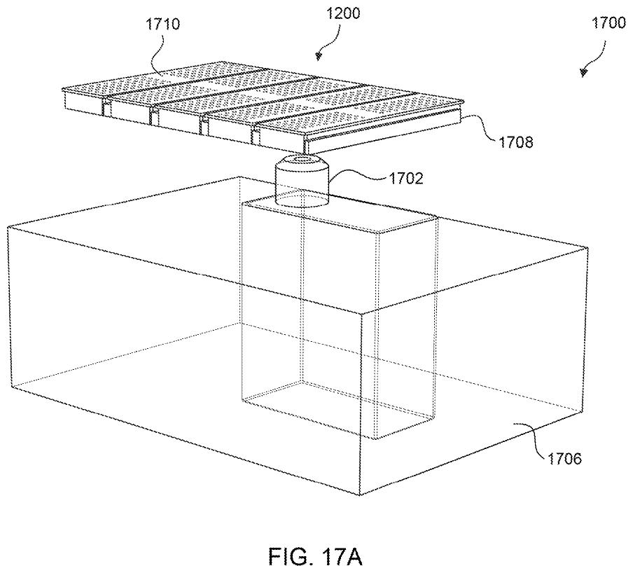

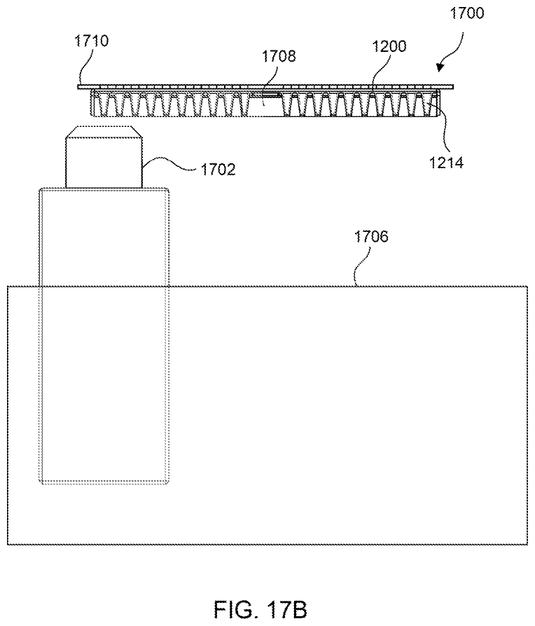

FIGS. 17A and 17B illustrate diagrams of an AST cartridge 1200 interfacing with an AST subsystem 1700 in the analyzer, according to embodiments of the present disclosure. AST subsystem 1700 represents an exemplary embodiment of AST subsystem 250 shown in FIG. 3B. In some embodiments, AST subsystem 1700 may be an imaging subsystem configured to perform optical interrogation of the enriched sample in the AST cartridge 1200. In some embodiments, the AST subsystem 1700 may comprise a microscope 1702, a scanning stage 1706, and a thermal block 1708.

In some embodiments, the AST cartridge 1200 may be placed in the thermal block 1708 of the AST subsystem 1700 by the pipettor (e.g., first or second pipettor 202, 204) in the analyzer device 200. In some embodiments, the thermal block 1708 may surround all sides of the reaction wells 1214 of the AST cartridge 1200 when the AST cartridge 1200 is positioned inside. In some embodiments, the thermal block 1708 may apply a 37° C. temperature control of the AST cartridge 1200. In some embodiments, a heating lid 1710 may be placed over the AST cartridge 1200 when positioned in the thermal block 1708 of the AST subsystem 1700. The microscope 1702 may be configured to obtain an optical readout of the bottom wall of each reaction well 1214 in the AST cartridge 1200 through the thermal block 1708.

In some embodiments, the scanning stage 1706 may comprise a motorized XYZ-translation stage that allows for motorized positioning of the microscope 1702 over the AST cartridge 1200. In some embodiments, the microscope 1702 may be configured to scan all of the reaction wells 1214 in the AST cartridge 1200 at one or more wavelengths (e.g., at two wavelengths) in less than about 300 seconds. In some embodiments, the microscope 1702 may use a 10× objective for optical readouts. In some embodiments, the microscope 1702 may use two optical channels for detecting different fluorescent signals (e.g., green and red fluorescence). In some embodiments, the microscope 1702 may scan the AST cartridge using two different wavelengths, such as wavelengths of 490 nm for excitation and 520 nm for emission (e.g., for detecting green fluorescence) and wavelengths of 540 nm for excitation and 620 nm for emission (e.g., for detecting red fluorescence).

In some embodiments, the microscope 1702 may be configured to perform fluorescence microscopy of the reaction wells 1214 in the AST cartridge 1200. In some embodiments, prior to acquiring one or more images using the microscope 1702, the first or second pipettor 202, 204 may dispense a first fluorescent dye and/or a second fluorescent dye to each reaction well 1214 in the AST cartridge 1200 to stain pathogens in the aliquots. In some embodiments, the first and/or second fluorescent dyes may be delivered to the reaction wells 1214 after incubation of the aliquots of the enriched sample in the reaction wells 1214 of the AST cartridge 1200. In some embodiments, the first fluorescent dye may comprise a DNA-binding dye that labels live cells in the aliquots of the reaction wells 1214, whereas the second fluorescent dye may comprise a fluorescent intercalating agent that cannot cross an intact cell membrane, and therefore only labels dead cells which have its membrane compromised in the aliquots of the reaction wells 1214. For example, the first fluorescent dye may be SYBR® Green or another fluorescent dye that emits green fluorescence (e.g., at emission wavelengths of about 500 to 560 nm), and the second fluorescent dye may be propidium iodide (PI) or another fluorescent agent that emits red fluorescence (e.g., at emission wavelengths of about 560 to 700 nm).

In some embodiments, the first or second pipettor 202, 204 may use jet dispensing to perform a contactless dispensing in order to avoid reaction well cross-contamination of the first fluorescent dye and/or the second fluorescent dye by the first or second needle 512 or 514 (coupled to the first or second pipettor 202, 204) penetrating respective septums 1208 of the reaction wells 1214 and without the first or second needle 512 or 514 contacting a bottom wall of each reaction well 1214. In some embodiments, a volume of the first fluorescent dye and/or the second fluorescent dye dispensed to each reaction well 1214 by the first or second needle 512 or 514 may be in a range of about 0.5 microliters to 10 microliters. In some embodiments, the microscope 1702 may perform fluorescence microscopy for image acquisition of the AST cartridge 1200 by acquiring one or more fluorescent images to detect the first fluorescent dye and/or the second fluorescent dye in each reaction well 1214 of the AST cartridge 1200. In some embodiments, the microscope 1702 may be configured to detect one fluorescent dye, two fluorescent dyes, or the like to analyze fluorescent images of the AST cartridge 1200.

In some embodiments, the microscope 1702 may be coupled to a processor (e.g., processing device 116) that is configured to perform image analysis and data processing of one or more images obtained of the AST cartridge 1200. In some embodiments, the processor coupled to the microscope 1702 may analyze the one or more images obtained of the AST cartridge 1200 by computing a number of fluorescent pathogens in each reaction well 1214 in the AST cartridge 1200. In some embodiments, the processor may determine whether a pathogen in a sample is resistant to various antimicrobials based on computing the number of live cells and the number of dead cells in reaction wells 1214 of the AST cartridge 1200 and applying various rules. For example, the processor may identify if a particular pathogen is resistant by determining whether a ratio of the number of dead cells (e.g., detected by red fluorescence in the one or more images) to the number of live cells (e.g., detected by green fluorescence in the one or more images) is below a predetermined threshold for a predetermined antimicrobial concentration.

In another example, the processor may identify if a particular pathogen is resistant by determining whether a ratio of the number of dead cells to the number of live cells is above a predetermined threshold for an aliquot of an enriched sample without the antimicrobial in the reaction well 1214. In another example, the processor may identify if a particular pathogen is resistant by determining whether a ratio of the number of live cells for an aliquot incubated with an antibiotic to the number of live cells for an aliquot incubated without the antibiotic is above a predetermined threshold. In yet another example, the processor may identify if a particular pathogen is resistant by determining whether a ratio of the number of live cells for an aliquot incubated with an antibiotic to the number of live cells for an aliquot at time zero (e.g., before incubation/reaction) is above a predetermined threshold. In yet another example, the processor may identify if a particular pathogen is resistant by determining whether a ratio of the brightness of live cells in the one or more images for an aliquot incubated with an antibiotic to the brightness of live cells in the one or more images for an aliquot at time zero (e.g., before incubation/reaction) is above a predetermined threshold. In some embodiments, the processor may apply one or more, or any combination of these example rules for identifying highly resistant pathogens and determining whether or not a particular pathogen is resistant to certain antimicrobials. In some embodiments, the microscope 1702 and the processor may acquire and process images of reaction wells 1214 where several replicates of each reaction (e.g., between an aliquot of an enriched sample and an antimicrobial) are performed in multiple reaction wells 1214 in the AST cartridge 1200.

In some embodiments, the processor may determine a minimum inhibitory concentration (MIC) of an antibiotic or antimicrobial that inhibits growth of a particular pathogen based on analyzing one or more images of the AST cartridge 1200. In some embodiments, different aliquots may be used to incubate the pathogens at different antibiotic concentrations. In some embodiments, the processor may determine the MIC as the minimum concentration of antibiotic at which the reaction has not shown a significant growth with respect to the initial number of pathogens. In some embodiments, the processor may determine the MIC by extrapolating the results for intermediate antimicrobial concentrations. In some embodiments, the processor may use the value obtained for the MIC to determine whether the pathogens are resistant, intermediate or susceptible based on a look-up table or a set of logical rules stored in a database (e.g., databases 110).

### Example Methods of Operation:

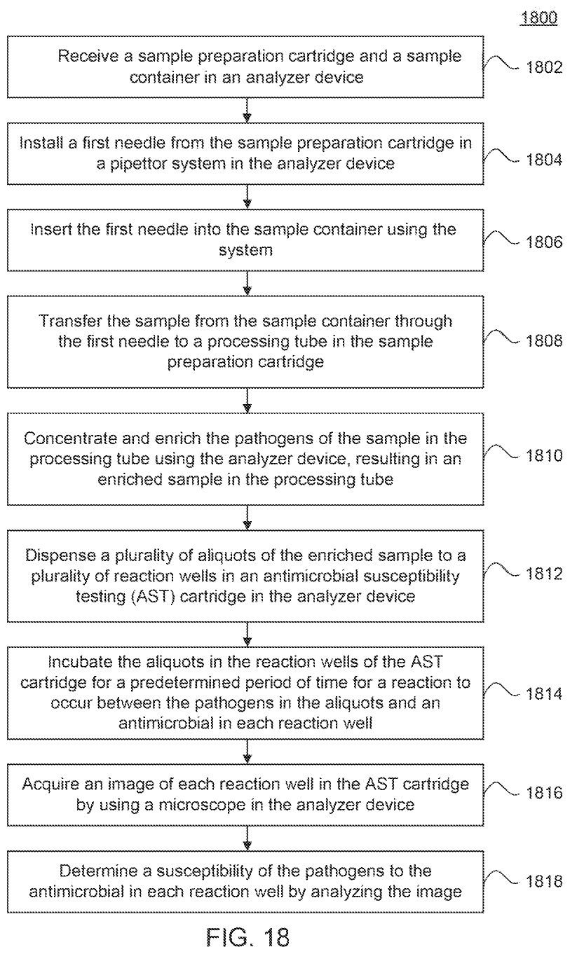

FIG. 18 illustrates a flowchart diagram of a method 1800 for performing AST of a sample, according to embodiments of the present disclosure. In some embodiments, method 1800 may describe the steps for performing AST using various components in the AST system, including analyzer 108, 200, sample preparation cartridge 114, 224, 500, processing tube 113, 510, 700, AST cartridge 115, 232, 1200, controller 109, and processing device 116, as discussed above with reference to FIGS. 1-17. It should be understood that the operations shown in method 1800 are not exhaustive and that other operations can be performed as well before, after, or between any of the illustrated operations. In various embodiments of the present disclosure, the operations of method 1800 can be performed in a different order and/or vary.

Method 1800 of FIG. 18 begins with step 1802, at which a sample preparation cartridge and a sample container are received in an analyzer device. In some embodiments, the analyzer device 200 may receive sample preparation cartridge 224 and sample container, which are placed in the sample cartridge drawer 220 and sample drawer 210, respectively, of the analyzer device 200, by a user or operator of the analyzer device 200. In some embodiments, the sample container may include a sample obtained from a patient, in which the sample comprises pathogens. In some embodiments, the sample in the sample container may comprise whole blood, urine, sterile body fluids, or other samples obtained from the patient.

At step 1804, a first needle from the sample preparation cartridge is installed in a pipettor system in the analyzer device. In some embodiments, needle 512 or 514 from the sample preparation cartridge 500 may be installed in the first or second pipettor 202, 204 by the pipette tip of the first or second pipettor 202, 204 moving above the sample preparation cartridge 500, pressing and fitting into a plastic body portion of the needle (e.g., plastic body 902 or 912).

At step 1806, the first needle is inserted into the sample container using the pipettor system. In some embodiments, after installation of the needle 512 or 514, the first or second pipettor 202, 204 may move above the sample container in the sample drawer 210 before pressing down and inserting the needle 512 or 514 into the sample container.

At step 1808, the sample from the sample container is transferred through the first needle to a processing tube in the sample preparation cartridge. In some embodiments, the needle 512 or 514 may draw up the sample into the plastic body reservoir of the first or second pipettor 202, 204, and the first or second pipettor 202, 204 may move to the sample preparation cartridge 500 to transfer the sample into a processing tube 510 in the sample preparation cartridge 500.

In order to transfer the sample from the plastic body reservoir of the first or second pipettor 202, 204 to the processing tube 510, the needle 512 or 514 (coupled to the first or second pipettor 202, 204) may pierce through a septum of the processing tube (e.g., septum 704 of processing tube 700). The first or second pipettor 202, 204 may then dispense the sample to the processing tube through the needle 512 or 514.

At step 1810, the pathogens of the sample are concentrated and enriched in the processing tube using the analyzer device, resulting in an enriched sample in the processing tube. In some embodiments, concentration and enrichment of the sample in step 1810 may use components in the analyzer device 200 to perform a series of steps, including centrifuging, removing fluid, adding growth media, and counting pathogens in an enriched sample.

At step 1812, a plurality of aliquots of the enriched sample are dispensed to a plurality of reaction wells in the AST cartridge in the analyzer device. In some embodiments, the first or second pipettor 202, 204 in the analyzer device 200 may dispense a plurality of aliquots of an enriched sample to the reaction wells 1214 in the AST cartridge 1200. In some embodiments, each aliquot may correspond to a respective reaction well 1214 in the AST cartridge 1200, and each reaction well 1214 may include an antimicrobial of a predetermined concentration for reacting with a respective aliquot of the enriched sample comprising pathogens.

At step 1814, the aliquots in the reaction wells of the AST cartridge are incubated for a predetermined period of time for a reaction to occur between the pathogens in the aliquots and an antimicrobial in each reaction well. In some embodiments, the first pipettor 202 may move the AST cartridge 1200 to the AST subsystem 250 or 1700, where the reaction wells 1214 may be placed in a thermal block or heater for temperature-controlled incubation. In some embodiments, the predetermined period of time for the incubation of the aliquots in the reaction wells 1214 is about two hours or less.

In some embodiments, after the incubating of the aliquots in each reaction well 1214, the first pipettor 202 may move the AST cartridge 1200 to a centrifuge (e.g., centrifuge 242 or 1412) in the analyzer device 200 to move the pathogens in each aliquot to a bottom wall of each reaction well 1214 for acquiring the image of the AST cartridge 1200. In some embodiments, after the incubating of the aliquots in each reaction well 1214, the first pipettor 202 may move the AST cartridge 1200 to the enrichment subsystem 1500, where a magnet (such as a magnet station) may be used to move the pathogens in each aliquot to a bottom wall of each reaction well 1214 for acquiring the image of the AST cartridge 1200. In some embodiments, the pathogens may be attached to magnetic beads in each aliquot.

At step 1816, an image of each reaction well in the AST cartridge is acquired by using a microscope in the analyzer device. In some embodiments, the first pipettor 202 may move the AST cartridge 1200 to the AST subsystem 1700, where the AST cartridge 1200 is imaged by microscope 1702 using scanning stage 1706. In some embodiments, each reaction well 1214 includes a bottom wall with an inner surface and an outer surface, in which the reaction between the pathogens in the aliquots and the antimicrobial in each reaction well 1214 occurs above the inner surface of the bottom wall of each reaction well 1214 in the AST cartridge 1200. In some embodiments, the image of the AST cartridge 1200 is acquired by microscope 1702 at the outer surface of the bottom wall of each reaction well 1214 in the AST cartridge 1200. In some embodiments, the microscope 1702 is configured to acquire one or more images taken from below the outer surface of the AST cartridge 1200. In some embodiments, the microscope 1702 may be configured to acquire one or more fluorescent images to detect a first fluorescent dye and/or a second fluorescent dye in each reaction well 1214 of the AST cartridge 1200.

At step 1818, a susceptibility of the pathogens to the antimicrobial in each reaction well is determined by analyzing the image. In some embodiments, a processor (e.g., processing device 116) coupled to the microscope 1702 in the analyzer device 200 may be used to determine susceptibility of the pathogens to the antimicrobial in each reaction well 1214 by analyzing one or more images obtained from the microscope 1702. In some embodiments, the processor may analyze the one or more images by computing a number of fluorescent pathogens in each reaction well 1212 in the AST cartridge 1200. In some embodiments, the processor may determine the susceptibility of the pathogens by determining whether one or more of the pathogens are susceptible, intermediate, or resistant to the antimicrobial depending on the outcome in each reaction well 1214 based on the staining of the cells in the aliquots by the first fluorescent dye.

In some embodiments, the processor may determine the susceptibility of the pathogens by determining whether one or more of the pathogens are susceptible, intermediate, or resistant to the antimicrobial in each reaction well 1214 based on the staining of the cells in the aliquots by the first fluorescent dye and/or the second fluorescent dye. In some embodiments, the processor may determine that a particular pathogen is resistant to a particular antimicrobial in a reaction well 1214 by determining that a ratio of a number of the dead cells to a number of the live cells is below a predefined threshold for the particular antimicrobial of a predetermined concentration based on the staining of the cells in the aliquots by the first fluorescent dye and/or the second fluorescent dye. In some embodiments, the processor may determine that a ratio of a number of the dead cells to a number of the live cells is below a predefined threshold of about 20% or about 10%.

In some embodiments, the processor coupled to the microscope 1702 may be configured to detect highly resistant pathogens based on an accelerated reaction in one or more reaction wells 1214 resulting from the usage of high dose antimicrobials in the reaction wells 1214. In particular, a first reaction well 1214 of the plurality of reaction wells 1214 may comprise a first concentration of the antimicrobial, wherein the first concentration is a high dose that is significantly higher than a clinical breakpoint for the antimicrobial and the pathogen. In some embodiments, a clinical breakpoint may represent a concentration or dose of an antimicrobial or antibiotic that is used to define whether an infection by a particular pathogen is susceptible to successful treatment with that antimicrobial or antibiotic. In some embodiments, using an antimicrobial concentration that is higher than the clinical breakpoint may accelerate a reaction between the antimicrobial and the concentrated pathogens in an aliquot in a reaction well. In some embodiments, by using the higher antimicrobial concentration, the processor may be configured to determine the susceptibility of the pathogens by comparing images of the first reaction well with a control to determine highly resistant pathogens in about one hour or less.

In addition to determining pathogen susceptibility, the processor may further be configured to determine a minimum inhibitory concentration (MIC) of a particular antimicrobial for inhibiting growth of a particular pathogen in the one or more pathogens based on the image of the AST cartridge 1200. Furthermore, the processor may be configured to determine whether the particular pathogen is susceptible, intermediate, or resistant to the particular antimicrobial based on a value of the MIC of the particular antimicrobial by parsing a set of rules in a database (e.g., databases 110) communicatively coupled to the processor. In some embodiments, the one or more databases 110 may be configured to store pathogen taxonomy data and/or outcomes from previous pathogen identification workflows (e.g., performed by analyzer device 200). In some embodiments, the processor may retrieve the pathogen taxonomy data and/or outcomes from the one or more databases 110 and use the retrieved data to determine the one or more rules that should be applied to perform resistant/intermediate/susceptible calls from the MIC.

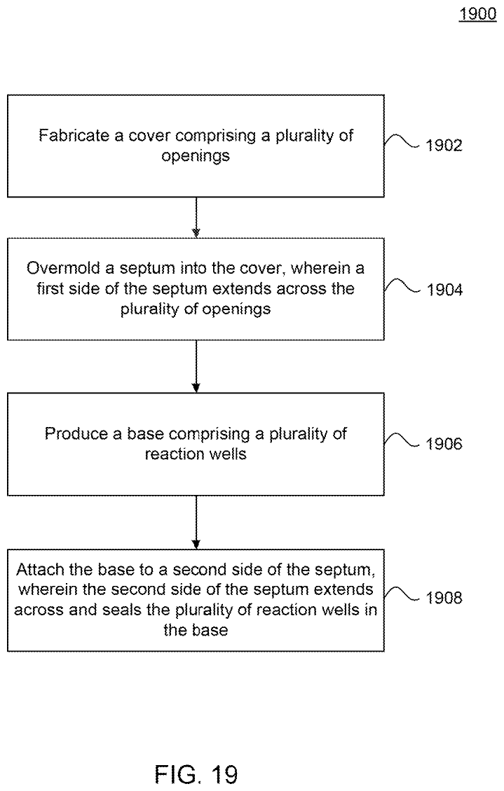

FIG. 19 illustrates a flowchart diagram of a method 1900 for manufacturing or assembling an AST cartridge, according to embodiments of the present disclosure. In some embodiments, method 1900 may describe the manufacturing and/or assembly of an AST cartridge, such as AST cartridge 232, 1200, as discussed above with reference to FIGS. 3B-17. It should be understood that the operations shown in method 1900 are not exhaustive and that other operations can be performed as well before, after, or between any of the illustrated operations. In various embodiments of the present disclosure, the operations of method 1900 can be performed in a different order and/or vary.

Method 1900 of FIG. 19 begins with step 1902, at which a cover comprising a plurality of openings is fabricated. In some embodiments, the cover 1202 of AST cartridge 1200 with the openings 1206 may be fabricated using injection molding. In some embodiments, the cover 1202 may be fabricated from polypropylene (PP), polycarbonate (PC), or another plastic material.

At step 1904, a septum is overmolded into the cover such that a first side of the septum extends across the plurality of openings in the cover. In some embodiments, the cover 1202 may be the substrate onto which the septum 1208 is molded directly on top of to create a single solid piece. In some embodiments, the septum 1208 may be made from a double layer of polytetrafluoroethylene (PTFE) and another material selected from the group consisting of silicone, rubber, and butyl rubber. In some embodiments, the septum 1208 may made from at least one of a rubber, polytetrafluoroethylene (PTFE), thermoplastic elastomer (TPE), silicone, butyl rubber, or a combination thereof.

At step 1906, a base comprising a plurality of reaction wells may be produced. In some embodiments, the base 1212 may be fabricated using injection molding. In some embodiments, the base 1212 may made from polystyrene (PS). In some embodiments, producing the base 1212 comprises producing the plurality of reaction wells 1214 connected together as a single component In some embodiments, the plurality of reaction wells 1214 in the base 1212 may be fabricated to have a conical shape. In some embodiments, a diameter of the bottom wall of each reaction well 1214 may be less than about 2 mm.

In some embodiments, an antimicrobial of a predetermined concentration may be added in a liquid form to each reaction well 1214 during manufacturing of the base 1212 and before attaching the base 1212 to the septum 1208. In some embodiments, the antimicrobial in each reaction well 1214 may be dried to obtain a dried or a freeze-dried form of the antimicrobial using forced air. In some embodiments, the steps of adding the antimicrobial and drying the antimicrobial in each reaction well 1214 may be completed within a predetermined period of time to prevent degradation of the antimicrobial. In some embodiments, the predetermined period of time for adding and drying the antimicrobial is about 10 minutes, 15 minutes, or the like.

At step 1908, the base may be attached to a second side of the septum, such that the second side of the septum extends across and seals the plurality of reaction wells in the base. In some embodiments, the first side of the septum 1208 is overmolded into the cover 1202, and the second side of the septum 1208 is attached to the base 1212 to provide a sealing mechanism over the reaction wells 1214 in the base 1212. In some embodiments, the base 1212 may be attached to the second side of the septum 1208 by using at least one of a snap-fit joint or a mechanical fastener. In some embodiments, each opening 1206 in the cover 1202 may be aligned with a respective reaction well 1214 of the plurality of reaction wells 1214 in the base 1212 during the attaching of the base 1212 to the second side of the septum 1208.

### Example Use Case

In some embodiments, the AST system described above can be used to determine a minimum inhibitory concentration for an antibiotic susceptibility test of a blood sample. In some embodiments, a blood sample is added to a reaction tube containing a lysis agent. In some embodiments, the amount of sample may be dependent on a size of reaction tube used. For example, if the reaction tube has a volume of 15 mL, then a sample volume in the tube will be less than 15 mL, such as 10 mL. In some embodiments, an additional agent, such as protease may be added to the reaction tube to improve the lysing action. For example, 100 μL-2 mL of protease may be added. To lyse and concentrate the sample, the samples may be incubated under low-speed rotary movement, and then centrifugated. A supernatant for the sample may be discarded until a preferred amount of the sample remains to prepare a matrix for the enrichment step. In some embodiments, a volume in the range of 200 μL-2 mL is kept after concentration. In some embodiments, a volume of about 500 μL is kept after concentration.

In some embodiments, enrichment is optionally performed. Prior to such enrichment a media broth may be added and vortexed. Enrichment may be carried out at an enrichment temperature with rotary agitation for a preferred period of time. In some embodiments, the total enrichment volume may be determined by the volume of media added to the volume of concentrated sample used. In some embodiments, the enrichment volume is about 5 mL (e.g., 500 μL concentrated sample+4500 μL media). A skilled artisan will recognize that other amounts and ratios may be used without departing from the present invention.

To separate the bacteria of interest from the blood in the sample, in some embodiments immunomagnetic separation may be used. For example, magnetic beads attached to biotinylated molecules relevant to the bacteria of interest may be used. These coated magnetic beads may be added to the concentrated sample in the reaction tube and incubated. In some embodiments, non-specific magnetic beads may be used to capture any bacteria available in the sample.

In some embodiments, after the capture of the bacteria with magnetic beads, a set of reagents may be used to digest, reduce and/or eliminate debris from the blood sample. As an example, proteases within the 2 μL-200 μL range may be used for this purposes, and/or DNAses to digest any cell-free DNA.

After incubation, collection of the magnetic beads may be performed by placing the reaction tube on or close to a magnet. Once the beads have collected in the portion of the reaction tube closest to the magnet, any supernatant may be pipetted off and discarded. The remaining beads, containing the sample bacteria, may be washed according to a cleaning process. Once the wash is complete, the beads may be resuspended with culture media. A desired amount of the beaded sample (e.g., 500 μL) may be extracted from the tube to a sample plate (such as a well plate), diluted as needed, and vortexed to achieve a desired volume and concentration of the sample for incubation, and delivered to a plurality of reaction wells in the plate. The sample plate may then be centrifuged to drive the bacteria to the bottom of the wells for imaging. In some embodiments, the sample plate is centrifuged for 1 minute at a spin of 3000 G.

The sample on the sample plate is then incubated for a desired amount of time. For example, the sample may be incubated for 5 hours. The sample plate may then be centrifuged to drive the bacteria to the bottom of the wells for imaging. The incubated sample may then be imaged through the bottom of the sample plate to determine the growth—and just the antimicrobial susceptibility—of the sample.

### EXPERIMENTAL EXAMPLES

As discussed below, several experiments were performed to test various embodiments of the antibiotic susceptibility test systems and methods described above.

### Example 1: Determination of Minimum Inhibitory Concentration of a Sample from a Blood Tube Sample

This example illustrates how to perform an antibiotic susceptibility test starting from a blood tube sample, according to embodiments disclosed herein.

First, Streptavidin coated beads were washed and immobilized. Beads attached to biotinylated molecules, such as antibodies in this case, had an overnight incubation with a phosphate saline buffer (adjusted to pH 7.4) at room temperature with gentle rotation (25 rpm). The typical binding capacity per mg (100 μL) of the magnetic beads used is approximately 20 μg of biotinylated antibody. After the coating, some washes with a phosphate saline buffer containing 0.01% of BSA (w/v) (adjusted pH 7.4) were performed. Finally, beads were resuspended to the desired concentration for the application. The antibody used was a biotin Klebsiella Polyclonal Antibody (PA1-73177, Invitrogen) and the magnetic beads used were Dynabeads MyOne Streptavidin T1 (65601, Thermofisher Scientific).

Blood samples were prepared from a single large blood pool from donors collected in 10 mL EDTA blood tubes which was spiked with the appropriate amount of bacteria (Klebsiella pneumoniae ATCC13883) depending on the target inoculum (around 100 CFU/10 mL). After the pool was spiked, 10 mL were transferred to a previously labelled 15 mL tube containing a lysing agent (700 μL of Isolator BC0507C, Oxoid) and an immiscible fluorocarbon oil (20 μL of Fluorinert™ FC-40 F9755, Sigma-Aldrich). To lyse and concentrate, samples were incubated for a short period of time (30 seconds) under rotatory movement at low speed (10 rpm) and centrifugated at 12.000 g force for 5 minutes with a fixed angle rotor. The supernatant of each sample was discarded until 500 μL used to prepare the matrix for the enrichment step. Half of the samples stopped processing at this point and these 500 μL were plated on agar plates as a check point to know how well the concentration process has gone and from how many CFU the next stage begins.

For the samples that continued with the experiment, prior to the enrichment step 4500 mL of media (Mueller Hinton broth) was added and a vortex of 1 minute was performed. This enrichment process was carried out at 37° C. and with rotary agitation at low speed (10 rpm) for 30 minutes. Once this stage was complete, a bank of dilutions was made and agar plates were plated to know the population of bacteria in each of the samples, know the replication time and the number of bacteria available for the immunomagnetic separation process.

For the immunomagnetic separation of the bacteria from the blood, 100 μL of antibody coated beads were added to each sample tube. The incubation conditions used were 30 minutes at 37° C. with a gentle rotary movement (25 rpm). After the incubation, to know how many bacteria were there (bound and not bound to the beads), a bank dilution using 100 μL of the matrix was plated on LB agar plates. The collection of the magnetic beads was performed by placing the tubes on the magnets for 15 minutes. After this time, the supernatant was pipetted off and discarded, and two steps of washes were carried out to clean the samples. Each washing step involved adding 5 mL of wash buffer (phosphate saline buffer, pH 7.4), mixing gently at 37° C. for 1 minute with a rotary movement (10 rpm), collecting the beads by placing the tubes on the magnet for a for a short period of time (1 minute), and pipetting off and discarding the supernatant. Once the washes were finished, the beads were resuspended with 500 μL of culture media (Mueller Hinton cation adjusted broth) and vortexed for 10-20 seconds. To determine the amount of bacteria captured by the magnetic beads, 100 μL of the final matrix were used to make a bank of dilutions and plated on LB agar plates that were grown at 37° C. for 16-18 hours. These samples were also observed under an inverted fluorescence microscope in order to know the bacteria present in the sample and calculate the amount needed to perform the AST test, which was the next and last step in this end-to-end assay. 5 μL of each sample were transferred to a 384-well plate with a flat polystyrene film bottom and incubated with fluorescence dyes (SYBR Green and Propidium Iodide) during 10 min at room temperature in the dark. The 384-well plate was centrifuged during 1 minute at 3000 g in a swinging-bucket movement and observed in an inverted fluorescence microscope. For each sample, a composite image covering the bottom of each well was constructed.

Based in that measurement, the sample was diluted (Mueller Hinton cation adjusted broth) to achieve a number of bacteria of approximately 4×10⁴ CFU/mL. The bacteria sample was incubated at a final concentration of 2×10⁴ CFU/mL at different Ciprofloxacin concentrations during 5 hours at 37° C. in a final volume of 10 μL. Ciprofloxacin concentrations tested were 0.125, 0.06, 0.03, 0.015, 0.008 and 0.004 mg/L. Three replicates were performed for each antibiotic concentration.

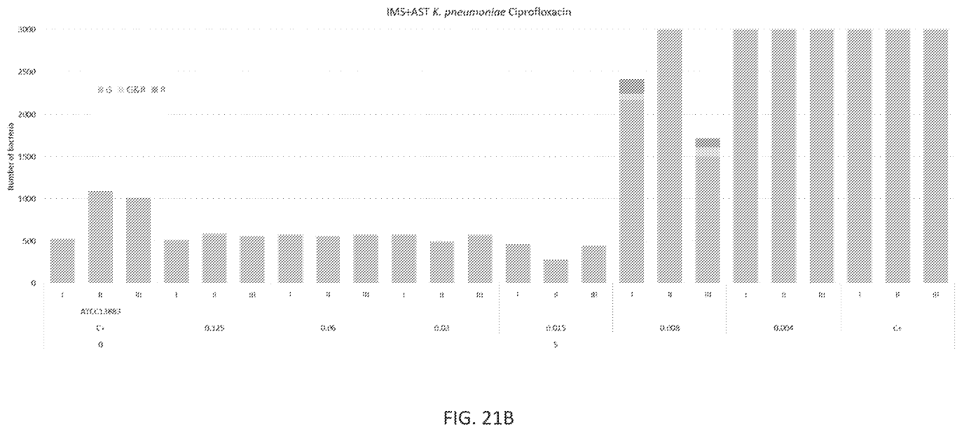

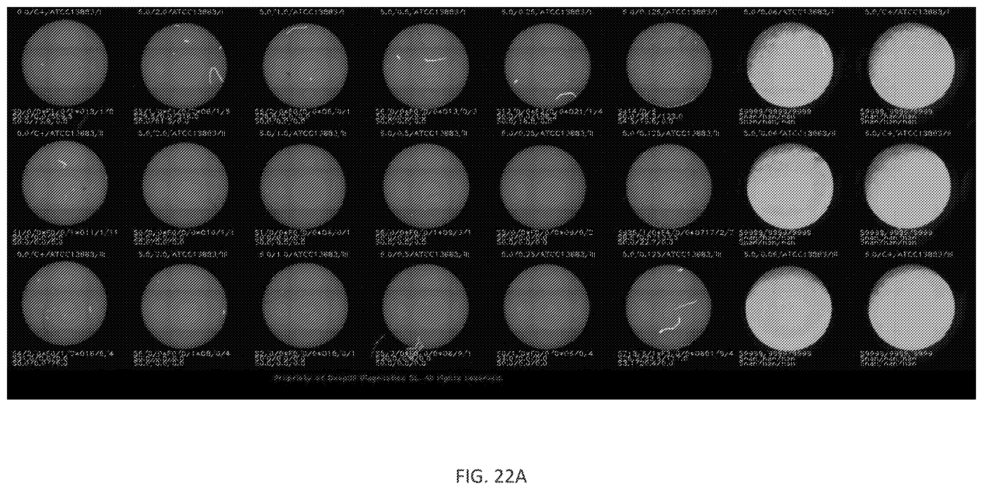

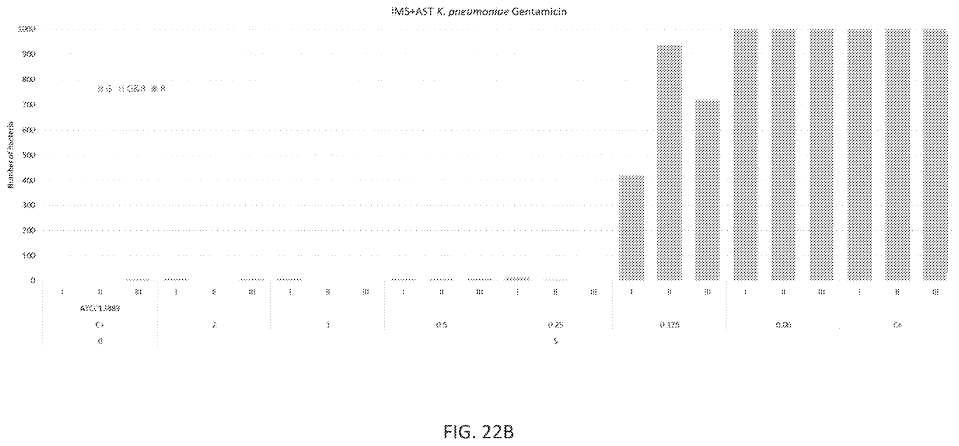

Samples were imaged at time 0 of incubation and after 5 hours of incubation (with and without antibiotic). 5 μL of each sample were transferred to a 384-well plate with flat polystyrene film bottom and incubated with fluorescence dyes (SYBR Green and Propidium Iodide) during 10 minutes at room temperature in the dark. The 384-well plate was centrifuged during 1 minute at 3000 g in a swinging-bucket centrifuge and observed in an inverted fluorescence microscope. For each sample a composite image was constructed. Images were analyzed with a custom software to count bacteria, detect filamentous bacteria, count red bacteria, and record the brightness of bacteria. These images are illustrated in FIG. 21A

Data obtained from the images was used to construct a Graph that showed growth at 0.008 and 0.004 mg/L of ciprofloxacin whereas at a concentration ≥0.015 mg/L did not grow (FIG. 21B). These results together with bacteria counting and data analysis represents an MIC of 0.015 mg/L for this strain, according to broth microdilution method this strain has an MIC of 0.03 mg/L, which accomplish the essential agreement. This graph is illustrated in FIG. 21B.

### Example 2: Determination of Minimum Inhibitory Concentration of a Sample from a Blood Culture Bottle

This example illustrates how to perform an antibiotic susceptibility test starting from a blood culture bottle, according to embodiments disclosed herein.

First, Streptavidin coated beads were washed and immobilized. The washing buffer and the immobilization process may be different depending on the application. Beads attached to biotinylated molecules, such as antibodies in this case, had an overnight incubation with a phosphate saline buffer (adjusted to pH 7.4) at room temperature with gentle rotation (25 rpm). The typical binding capacity per mg (100 μL) of magnetic beads used is approximately 20 μg of biotinylated antibody. After the coating, some washes were performed with a phosphate saline buffer containing 0.01% of BSA (w/v) (adjusted pH 7.4) and then beads were finally resuspended to the desired concentration for the application. The antibody used was a biotin Klebsiella Polyclonal Antibody (PA1-73177, Invitrogen) and the magnetic beads used were Dynabeads MyOne Streptavidin T1 (65601, Thermofisher Scientific).

Blood samples were prepared from a previously mixed single large blood pool which was spiked with the appropriate amount of bacteria (Klebsiella pneumoniae ATCC13883) depending on the target inoculum (around 100 CFU). After the pool was spiked, 10 mL were transferred to a previously labelled blood culture system (442023, BD BACTEC™ Plus Aerobic/F) and mixed with an agitation platform (5 minutes at 150 rpm). Once the time had elapsed, a reasonable time was waited (30 seconds-1 minute) for the resins to precipitate. A 5 mL sample was then extracted.

The immunomagnetic separation of the bacteria was performed using a 15 mL tube containing 5 mL of a matrix extracted from a blood culture bottle and 100 μL of beads coated with anti-K. pneumoniae antibodies. The incubation conditions used were 30 minutes at 37° C. and with a gentle rotary movement (25 rpm). After the incubation, a bank of dilution using 100 μL of the matrix and plating were made in order to know how many bacteria were contained in each sample (bacteria bound and not bound to the beads). The collection of the magnetic beads was performed by placing the tubes on the magnets for 7.5 minutes. After this time, the supernatant was pipetted off and discarded, and three steps of washes were carried out to clean the samples. Each washing step involved adding 5 mL of culture media (Mueller Hinton cation adjusted broth), vortexing 1 minute, mixing gently at 37° C. for 2 minutes with a slow rotary movement (10 rpm), collecting beads by placing the tubes on the magnet for a short period of time (2 minutes) and pipetting off and discarding the supernatant. After washes, the beads were resuspended with 500 μL of culture media (Mueller Hinton cation adjusted broth) and vortexed for 1 minute. To determine the amount of bacteria captured by the antibody coated magnetic beads, 100 μL of the final matrix were used to make a bank of dilutions and plated on LB agar plates which were grown at 37° C. for 16-18 hours. The samples were also observed under an inverted fluorescence microscope in order to determine the number of bacteria present in the sample and calculate the adjustment needed to perform the AST test. 5 μL of each sample were transferred to a 384-well plate with flat polystyrene film bottom and incubated with fluorescence dyes (SYBR Green and Propidium Iodide) during 10 minutes at room temperature in the dark. The 384-well plate was centrifuged during 1 minute at 3000 g in a swinging-bucket centrifuge and observed in an inverted fluorescence microscope. For each sample a composite image covering the bottom of each well was constructed. Images were analyzed with a custom software to determine the number of bacteria.

Based in that measurement, the sample was diluted (Mueller Hinton cation adjusted broth) to achieve a number of bacteria of approximately 4×10⁴ CFU/mL. The bacteria sample was incubated at a final concentration of 2×10⁴ CFU/mL at different Gentamicin concentrations during 5 hours at 37° C. in a final volume of 10 μL. Gentamicin concentrations tested were 2, 1, 0.5, 0.25, 0.125 and 0.06 mg/L. Three replicates were performed for each antibiotic concentration.

Samples were analyzed at time 0 of incubation and after 5 h incubation (with and without antibiotic). 5 μL of each sample were transferred to a 384-well plate with flat polystyrene film bottom and incubated with fluorescence dyes (SYBR Green and Propidium Iodide) during 10 minutes at RT in the dark. The 384-well plate was centrifuged during 1 minute at 3000 g in a swinging-bucket movement and observed in an inverted fluorescence microscope. For each sample a composite image was constructed. Images were analyzed with a custom software that to count bacteria, detect filamentous bacteria, count red bacteria, and record the brightness of bacteria. These images are illustrated in FIG. 22A.

Data obtained from the images was used to construct a Graph that showed growth at 0.125 and 0.06 mg/L of gentamicin, whereas at a concentration ≥0.25 did not grow (FIG. 22B). These results together with bacteria counting and data analysis represents an MIC of 0.25 mg/L for this strain, according to broth microdilution method this strain has an MIC of 0.5-0.25 mg/L. This graph is illustrated in FIG. 22B.

### Example 3: Determination of Minimum Inhibitory Concentration for Gram-Negative Bacteria to Gentamicin

The minimal inhibitory concentration was determined using embodiments disclosed herein for one strain of Gram-negative bacteria Klebsiella pneumoniae (ATCC13883) that is susceptible to gentamicin with an MIC of 0.25-0.5 mg/L according to the broth microdilution method. Bacteria was grown in a liquid culture until an exponential phase in culture media. The cultures were diluted in culture media (Mueller Hinton cation adjusted broth) and was incubated at a final concentration of 2×10⁴ CFU/mL during 5 hours at 37° C. at different concentrations of gentamicin in a final volume of 10 μL. Gentamicin concentrations tested were 4, 2, 1, 0.5, 0.25 and 0.125 mg/L. Three replicates were performed for each antibiotic concentration.

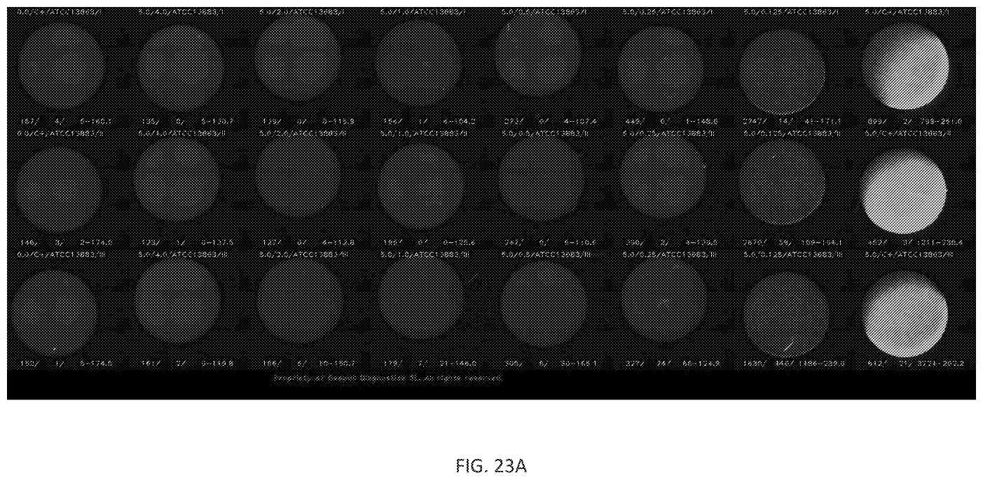

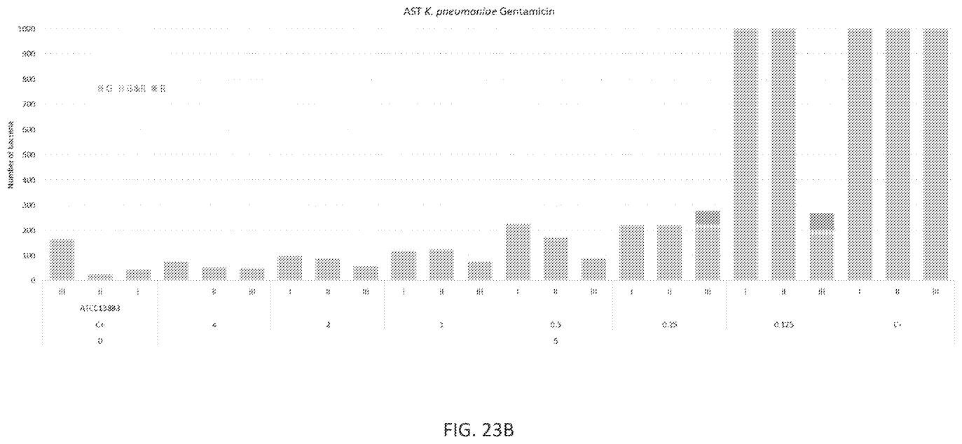

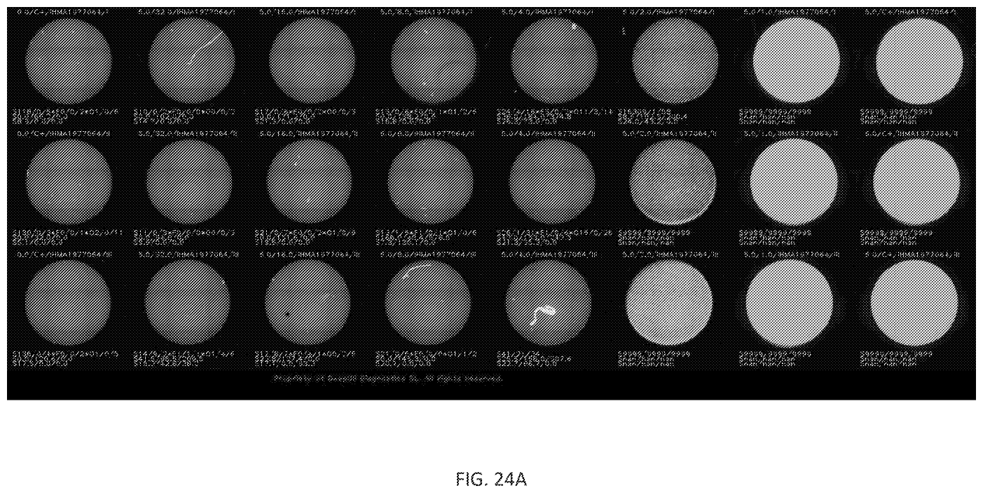

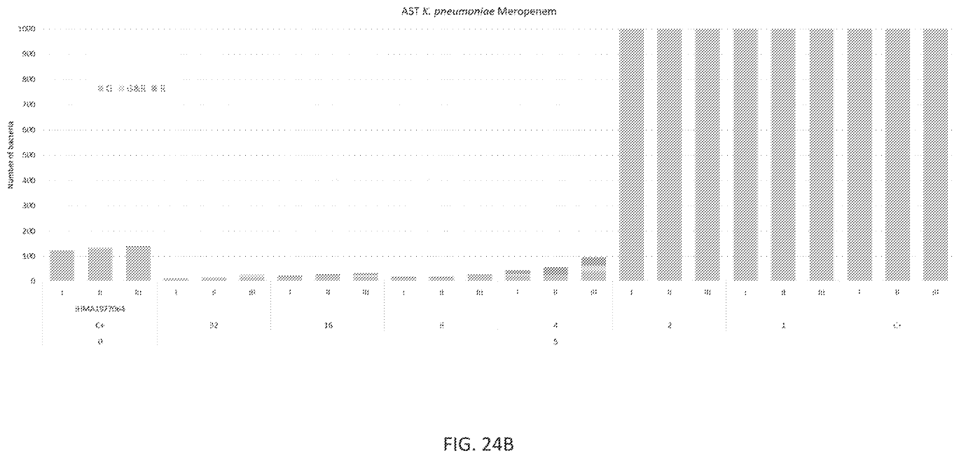

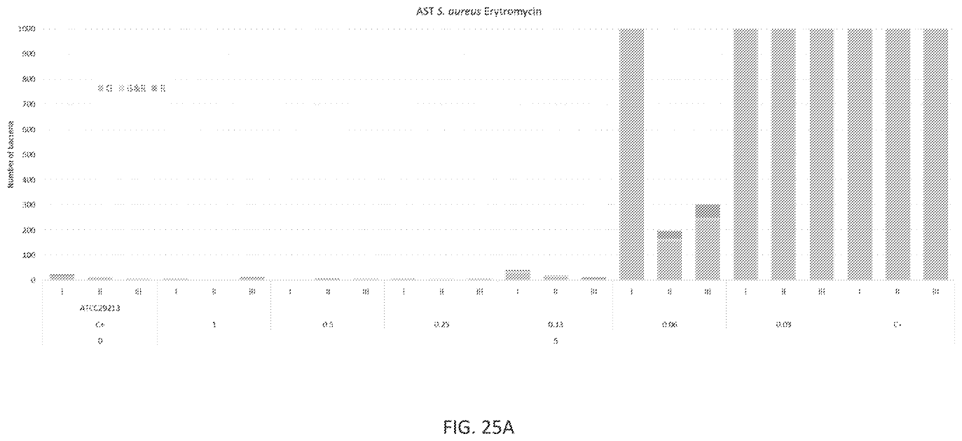

Samples were analyzed at time 0 of incubation and after 5 hours incubation (with and without antibiotic). Samples were incubated with fluorescence dyes (SYBR Green and Propidium Iodide) during 10 minutes at room temperature in the dark. After dye incubation, a sample of 1 μL was diluted with 4 μL of buffer (NaCl 0.9%) and transferred to a 384-well plate with flat polystyrene film bottom. The 384-well plate was centrifuged during 1 minute at 3000 g in a swinging-bucket movement and observed in an inverted fluorescence microscope. For each sample, a composite image covering the bottom of each well was constructed. Images were analyzed with a custom software to count bacteria, detect filamentous bacteria, counts red bacteria, and record the brightness of bacteria. These images are illustrated in FIG. 23A

Data obtained from the images was used to construct a Graph that showed bacterial growth at 0.125 mg/L of gentamicin, whereas at a concentration ≥0.25 mg/L did not grow (FIG. 23B). These results together with bacteria counting and data analysis represents an MIC of 0.25 mg/L for this strain which corresponds with the MIC obtained by the broth microdilution method. The results show that with 5 hours incubation at different antibiotic concentrations and using bacterial growth and bacteria characteristics it is possible to determine the MIC of these Gram-negative strains to gentamicin. This graph is illustrated in FIG. 23B.

### Example 4: Determination of Minimum Inhibitory Concentration for Gram-Negative Bacteria to Meropenem

The minimal inhibitory concentration was determined using embodiments disclosed herein for one strain of Gram-negative bacteria Klebsiella pneumoniae (IHMA1977064) with an MIC of 8 mg/L to meropenem according to the broth microdilution method. Bacteria was grown in liquid culture until an exponential phase in culture media. The cultures were diluted in culture media (Mueller Hinton cation adjusted broth) and was incubated at a final concentration of 2×10⁴ CFU/mL during 5 hours at 37° C. at different concentrations of meropenem in a final volume of 10 μL. Meropenem concentrations tested were 32, 16, 8, 4, 2 and 1 mg/L of gentamicin. Three replicates were performed for each antibiotic concentration.

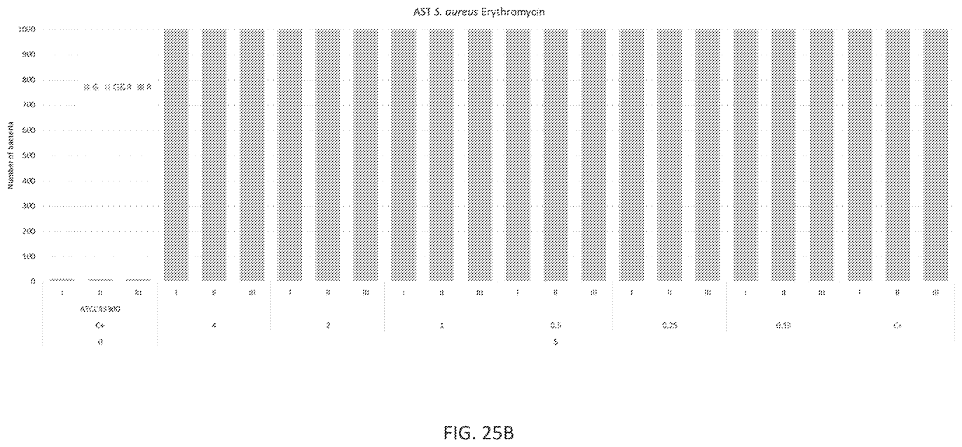

Samples were analyzed at time 0 of incubation and after 5 hours incubation (with and without antibiotic). 5 μL of each sample were transferred to a 384-well plate with flat polystyrene film bottom and incubated with fluorescence dyes (SYBR Green and Propidium Iodide) during 10 minutes at room temperature in the dark. The 384-well plate was centrifuged during 1 minute at 3000 g in a swinging-bucket movement and observed in an inverted fluorescence microscope. For each sample, a composite image covering the bottom of each well was constructed. Images were analyzed with a custom software to count bacteria, detect filamentous bacteria, counts red bacteria, and record the brightness of bacteria. These images are illustrated in FIG. 24A.

Data obtained from the images was used to construct a Graph that showed bacterial growth at 2 and 1 mg/L of meropenem, whereas at a concentration ≥4 mg/L did not grow (FIG. 24B). These results together with bacteria counting and data analysis represents an MIC of 4 mg/L, according to broth microdilution method this strain has an MIC of 8 mg/L, which accomplish the essential agreement. This graph is illustrated in FIG. 24B.

The results show that with 5-hour incubation at different antibiotic concentrations and using bacterial growth and bacteria characteristics, it is possible to determine the MIC of these Gram-negative strains to meropenem.

### Example 5: Determination of Minimum Inhibitory Concentration for Gram-Positive Bacteria to Erythromycin

The minimal inhibitory concentration was determined for two strains of Gram-positive bacteria Staphylococcus aureus, one susceptible (ATCC29213) and one resistant (ATCC43300) to erythromycin, according to embodiments described herein. The MIC of these strains was obtained by broth microdilution method, S. aureus susceptible to erythromycin had an MIC of 0.25-0.5 mg/L, and the resistant strain an MIC≥512 mg/L. Bacterial strains were grown in liquid culture until an exponential phase in rich media. Bacteria cultures were diluted in culture media (Mueller Hinton cation adjusted broth) and were incubated at a final concentration of 2×10⁴ CFU/mL during 5 hours at 37° C. at different concentrations of gentamicin in a final volume of 10 μL. S. aureus strain susceptible to erythromycin was tested at 1, 0.5, 0.25, 0.125, 0.06 and 0.03 mg/L, whereas resistant strain was tested at 4, 2, 1, 0.5, 0.25 and 0.125 mg/L of erythromycin. Three replicates were performed for each antibiotic concentration.

Samples were analyzed at time 0 of incubation and after 5 hours incubation (with and without antibiotic). Samples were incubated with fluorescence dyes (SYBR Green and Propidium Iodide) during 10 minutes at room temperature in the dark. After dye incubation a sample of 1 μL was diluted with 4 μL of buffer (NaCl 0.9%) and transferred to a 384-well plate with flat polystyrene film bottom. The 384-well plate was centrifuged during 1 minute at 3000 g in a swinging-bucket movement and observed in an inverted fluorescence microscope. For each sample, a composite image covering the bottom of each well was constructed. Images were analyzed with a custom software to count bacteria, detect filamentous bacteria, counts red bacteria, and record the brightness of bacteria.

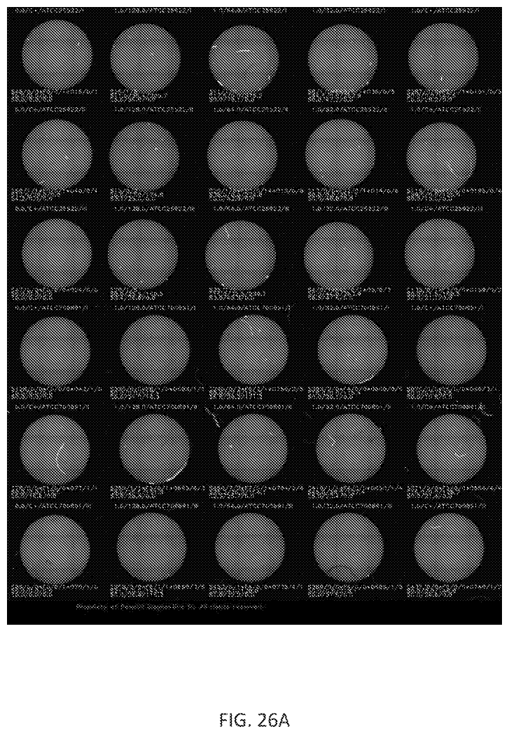

Data obtained from images was used to perform a Graph either for S. aureus susceptible and resistant to erythromycin (FIGS. 25A and 25B, respectively). S. aureus susceptible to erythromycin showed growth at 0.125 mg/L of erythromycin, whereas at a concentration ≥0.25 mg/L did not grow (FIG. 25A). These results together with bacteria counting and data analysis represents an MIC of 0.25 mg/L for this strain which corresponds with the MIC obtained by broth microdilution method. Data analysis of S. aureus strain resistant to erythromycin (FIG. 25B) showed growth at 4 mg/L and lower concentrations of erythromycin, which indicates an MIC≥4 mg/L for this strain. The MIC obtained by broth microdilution method was ≥512 mg/L which indicates that with both methods this strain is resistant to erythromycin (EUCAST and CLSI breakpoints indicates that strains of S. aureus with an MIC≥2 to erythromycin are resistant). The results show that with 5 hours incubation at different antibiotic concentrations and using bacterial growth and bacteria characteristics it is possible to determine the MIC of these Gram-positive strains to erythromycin.

### Example 6: Fast Determination of Resistant Bacteria to Ampicillin

To rapidly detect high resistant bacteria to a particular antimicrobial agent, bacteria were grown during a short period of time (1 hour) in the presence of high dose of that antimicrobial agent. Two strains of Escherichia coli, one susceptible (ATCC25922) and the other resistant (ATCC700891) to ampicillin were grown until an exponential phase in culture media. Bacteria cultures were diluted in culture media (Mueller Hinton cation adjusted broth) and were incubated at a final concentration of 2×10⁴ CFU/mL during 1 hour at 37° C. in the presence of ampicillin at a final concentration of 128 mg/L, 64 mg/L, and 32 mg/L in a final volume of 10 μL. Three replicates were performed for each antibiotic concentration.

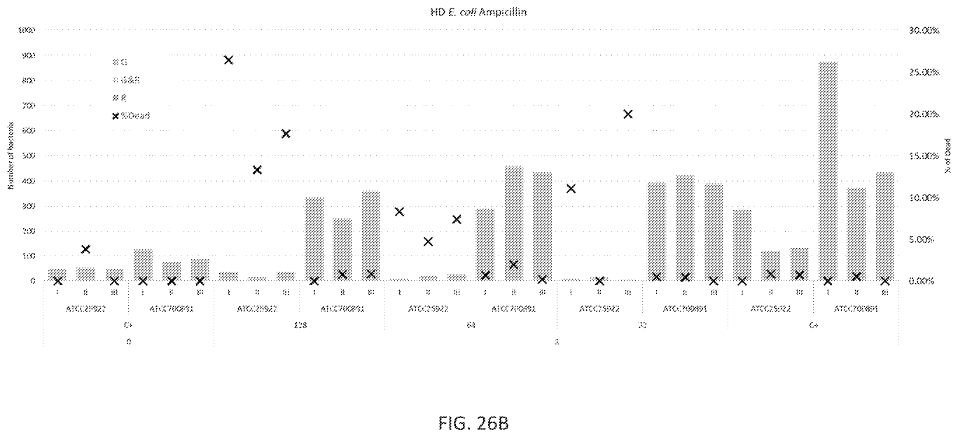

Samples were visualized at time 0 that represents bacteria before any incubation and after 1 hour incubation. 5 μL of each sample were transferred to a 384-well plate with flat polystyrene film bottom and incubated with fluorescence dyes (SYBR Green and Propidium Iodide) during 10 minutes at room temperature in the dark. The 384-well plate was centrifuged during 1 minute at 3000 g in a swinging-bucket movement and observed in an inverted fluorescence microscope. For each sample a composite image covering the bottom of each well was constructed. Images were analyzed with a custom software to count bacteria, detect filamentous bacteria, counts red bacteria, and record the brightness of bacteria. These images are illustrated in FIG. 26A.

When comparing images of both E. coli strains at a different ampicillin concentration, the E. coli strain susceptible to ampicillin with an MIC of 4 mg/L according to broth microdilution method, does not grow at any ampicillin concentration tested (FIG. 26B), bacteria are in filamentous form in the three concentrations tested and there are also the presence of red bacteria at 128 mg/L of ampicillin, which indicates that bacteria were dead. Whereas the E. coli strain resistant to ampicillin with an MIC≥128 mg/L according to broth microdilution method, showed growth after 1 hour at all three tested ampicillin concentration without presence of filamentous bacteria or red bacteria (FIG. 26B).

The results demonstrate that with only 1 hour of incubation at a high dose antibiotic concentration and using bacterial growth and bacterial characteristics, it is possible to detect resistant bacteria to an ampicillin.

### Example 7: Blood Sample Clean-Up with Specific Beads

Different time conditions were evaluated to establish the optimal duration of the capture of the bacteria by the magnetic beads. First, streptavidin coated beads were washed with a washing buffer based on the application. Once washed, the beads were immobilized. The beads were attached to biotinylated molecules, such as antibodies like in this case, performing an overnight incubation with phosphate saline buffer (adjusted to pH 7.4) at room temperature with gentle rotation (25 rpm). The typically binding capacity per mg (100 μL) of the magnetic beads tested was approximately 20 μg of biotinylated antibody. After completing the coating, some washes with a phosphate saline buffer containing 0.01% of BSA (w/v) (adjusted to pH 7.4) were performed, and finally the beads were resuspended to the desired concentration for the application. The antibody used was a biotin Klebsiella Polyclonal Antibody (PA1-73177, Invitrogen) and the magnetic beads used were Dynabeads MyOne Streptavidin T1 (65601, Thermofisher Scientific).

Different capture times were evaluated with blood samples. Blood samples were prepared from a single large pre-shaken blood pool which was spiked with the appropriate amount of bacteria (Klebsiella pneumoniae ATCC13883) depending on the target inoculum. After the pool was spiked, 10 mL were transferred to a previously labelled blood culture system (442023, BD BACTEC™ Plus Aerobic/F) and mixed with an agitation platform (5 minutes at 180 rpm). Once the time has elapsed, a reasonable time (30 seconds-1 minute) was waited for the resins to precipitate and a 5 mL sample was extracted. No more than 10 mL was extracted from each blood culture system.

The 5 mL of blood that came from the blood culture system was transferred to a 15 mL tube and the required amount of antibody coated beads (100 μL) were added. The incubation was performed for 30 minutes at 37° C. with a gentle rotary movement (25 rpm). After incubation, a bank of dilution using 100 μL of the matrix was performed and it was plated. The beads were collected by placing the tubes on the magnet for the tested times (15-7.5 minutes) and then the supernatant was pipetted off and discarded. At that point, 5 mL of washing buffer were added to start the three corresponding batches of washes. Each washing step included vortexing for 1 minute and incubating samples at 37° C. for 2 minutes with a gentle rotary movement (25 rpm), collecting the beads by placing the tubes on the magnet for a short period of time (5-2.5 minutes) and pipetting off and discarding the supernatant. After the washes, the beads were resuspended with 500 μL of culture media (Mueller Hinton cation adjusted broth) and vortexed 1 minute. The next step included performing a bank of dilution using 100 μL of the matrix and plating it. In parallel, these samples were also visualized under the microscope to check if there was an agreement between the two types of readings (plates versus microscope). To observe the samples under an inverted fluorescence microscope, 5 μL of each sample were transferred to a 384-well plate containing fluorescence dyes (SYBR-Green+Propidium Iodide). Subsequently, this plate was incubated for 10 minutes at room temperature and a centrifugation process was carried out (3000 g force for 1 minute) before its visualization. Table 1 below shows that the results have little dependency on the capture time:

TABLE 1

| IMS | Wash | | | | | |
|---|---|---|---|---|---|---|
| magnet | magnet | | Plates | | Microscope | |
| time | time | Initial | Counts | Recovery | Counts | Recovery |
| (min) | (min) | (CFU) | (CFU) | values (%) | (CFU) | values (%) |
| 15 | 5 | 173000 | 146000 | 84.39 | 208875 | 120.74 |
| | | 154000 | 148625 | 96.51 | 167130 | 108.53 |
| 7.5 | 5 | 195750 | 175500 | 89.66 | 282935 | 144.54 |
| | | 141500 | 98750 | 69.79 | 144970 | 102.45 |
| 15 | 2.5 | 270875 | 106000 | 39.13 | 149380 | 55.15 |
| | | 139375 | 136750 | 98.12 | 223845 | 160.61 |
| 7.5 | 2.5 | 112875 | 160000 | 141.75 | 183060 | 162.18 |
| | | 138750 | 165250 | 119.10 | 223600 | 161.15 |

### Example 8: Blood Sample Clean-Up Process with Non-Specific Beads

A series of reagents to improve sample clean-up from cellular debris derived from blood cell lysates have been evaluated. Blood samples were prepared from a single large pre-shaken blood (5 minutes at 150 rpm). After shaking, 10 mL were transferred to a previously labelled blood culture system (442023, BD BACTEC™ Plus Aerobic/F) and mixed with an agitation platform (5 minutes at 180 rpm). Once the time has elapsed, a reasonable time was waited (30 seconds-1 minute) for the resins to precipitate and a 10 mL sample was extracted. No more than 10 mL was extracted from each blood culture system.

10 mL of blood samples extracted from the blood culture system were transferred to a 15 mL tube to be lysed with a variable volume of lysing agent (700 μL of 2.8 g/100 mL of saponin S4521, Sigma-Aldrich). The tube used for lysis and concentration step also contained an immiscible fluorocarbon oil (20 μL of Fluorinert™ FC-40 F9755, Sigma-Aldrich). Apart from the lysis agent, other reagents such as protease were also added to increase their action (250 μL of Protease from Aspergillus oryzae P6110, Sigma-Aldrich). To perform the lysis and concentration steps, it was necessary to incubate samples for a short period of time (30 seconds) under rotatory movement at low rpm (10 rpm) and then centrifugate at 12000 g force for 5 minutes to discard the supernatant until 500 μL to prepare the matrix for the IMS that includes 500 μL of TTGB 2× buffer. Once the initial matrix was obtained, other complementary reagents such as DNAses (different concentration and time) were added to improve the elimination of cell debris. Prior to the addition of the magnetic beads, the samples were spiked with the appropriate amount of bacteria (Escherichia coli ATCC25922) depending on the target inoculum. After the matrix was spiked, a short vortex was done. It is at this point that the magnetic beads were added, but not before having been gently mixed.

The magnetic beads used were bound to the peps6 peptide, a synthetic version of the ApoH protein, a blood-borne human protein that is considered as a potentially infectious product (MP10031, ApoH Technologies). The conditions to perform the immunomagnetic separation with these beads were 30 minutes at 37° C., with a gentle rotary movement (25 rpm) and 20 degrees of inclination. Once binding occurred, 4 mL of 1×TTGB buffer were added to use 100 μL of the sample to perform a dilution bank and an agar plate was used to determine the number of bacteria before magnetic retention. The necessary time for a correct magnetic separation was established in approximately 15 minutes. Subsequently, the supernatant was eliminated, and 4 wash batches were carried out. Each step of wash included adding 5 mL of wash buffer (Mueller Hinton cation adjusted broth), vortexing for 1 minute, incubating the sample at 37° C. for 2 minutes with a gentle rotary movement (25 rpm), collecting the beads by placing the tubes on the magnet for a short period of time (5 minutes) and pipetting off and discarding the supernatant.

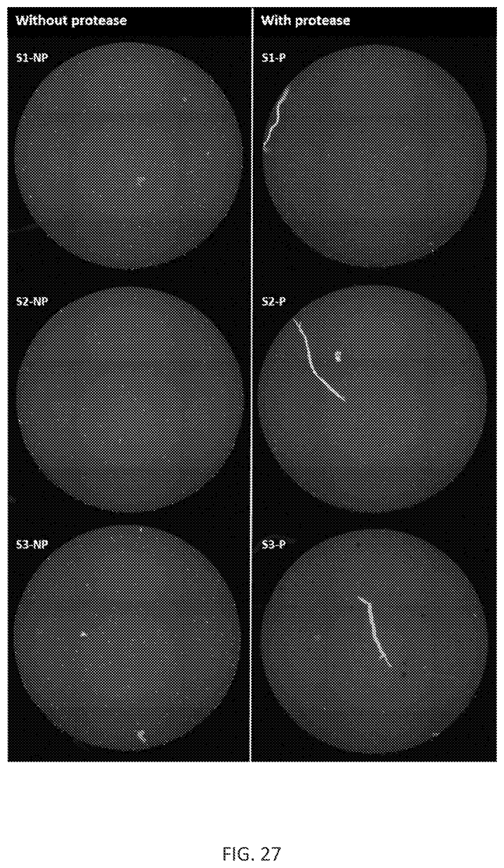

After the washes, the beads were resuspended with 500 μL of culture media (Mueller Hinton cation adjusted broth), vortexed for 1 minute, and 250 μL of each sample were transferred to a two 1.5 mL tube. At this point, the second treatment with reagents was carried out to further clean the sample and disaggregate possible aggregates that had been detected in previous assays. Specifically, 10 μL of protease were added and left to act for 30 seconds. To avoid the action of the protease on the bacteria, it was eliminated by centrifugation for 1 minute at 1000 g forces with a fixed-angle rotor. Finally, the supernatant was discarded, and beads resuspended with 500 μL of (Mueller Hinton cation adjusted broth) and vortexed for 1 minute. A bank dilution using 100 μL of the matrix and agar plate of all sample were performed and samples were also observed under a microscope. FIG. 27 illustrates images of samples with and without protease.

5 μL of each sample were transferred to a 384-well plate containing a quantity of dye (SYBR-Green+PI), incubated for 10 minutes at room temperature and a centrifugation process was carried out (3000 g force for 1 minute) before its visualization.

Table 2 below shows that a significant portion of bacteria is recovered at the end of the clean-up process, and that the number of counts is higher when proteases are used after washes, which is probably due to the disaggregation of bacteria aggregates.

TABLE 2

| | Protease | | Plates | | Microscope | |
|---|---|---|---|---|---|---|
| | after | Initial | Counts | Recovery | Counts | Recovery |
| Sample | washes | (CFU) | (CFU) | values (%) | (CFU) | values (%) |
| S1-NP | No | 40500 | 109000 | 269.14 | 92300 | 227.90 |
| S1-P | Yes | 40500 | 86125 | 212.65 | 145800 | 360.00 |
| S2-NP | No | 37500 | 95750 | 255.33 | 59700 | 159.20 |
| S2-P | Yes | 37500 | 91625 | 244.33 | 128600 | 342.93 |
| S3-NP | No | 42000 | 107125 | 255.06 | 109000 | 259.52 |
| S3-P | Yes | 42000 | 110000 | 261.90 | 148200 | 352.86 |

### Example Computer System:

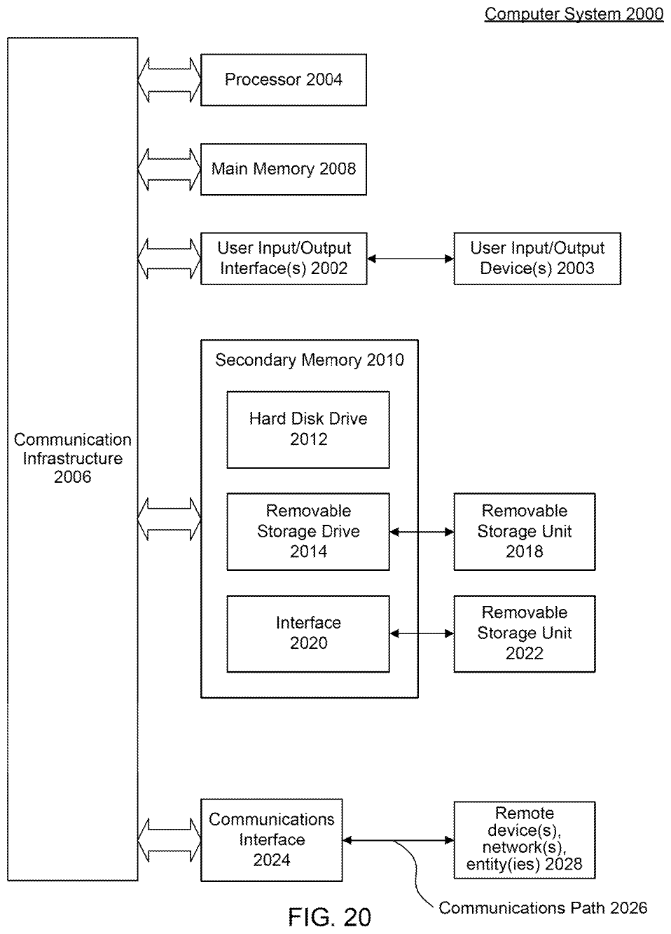

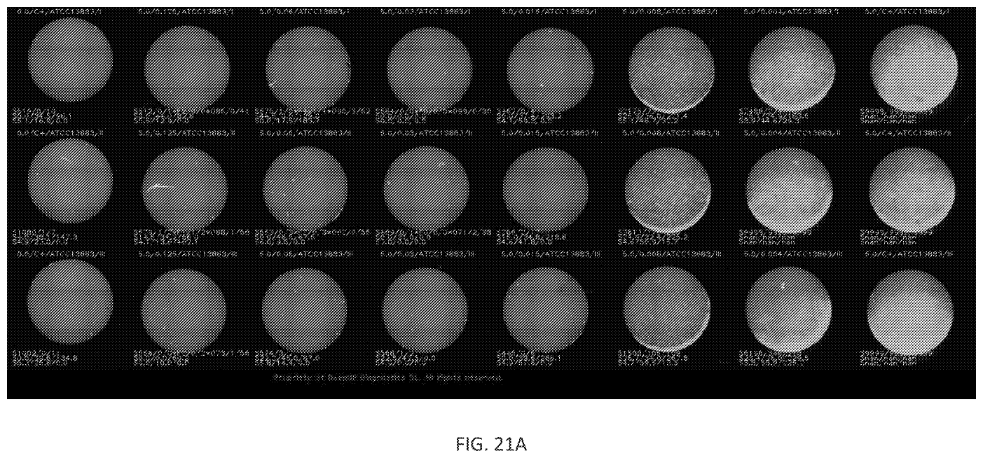

FIG. 20 is a block diagram of example components of computer system 2000. One or more computer systems 2000 may be used, for example, to implement any of the embodiments discussed herein, as well as combinations and sub-combinations thereof. In some embodiments, one or more computer systems 2000 may be used to perform image acquisition, image analysis, and data processing, such as for the microscope in the AST subsystem 1700, or the processing device 116, as described herein. In some embodiments, one or more computer systems 2000 may also be used in the controller 109 for programming and operating movements of various components in the analyzer device 200. Computer system 2000 may include one or more processors (also called central processing units, or CPUs), such as a processor 2004. Processor 2004 may be connected to a communication infrastructure or bus 2006.

Computer system 2000 may also include user input/output interface(s) 2002, such as monitors, keyboards, pointing devices, etc., which may communicate with communication infrastructure 2006 through user input/output device(s) 2003.

One or more of processors 2004 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 2000 may also include a main or primary memory 2008, such as random access memory (RAM). Main memory 2008 may include one or more levels of cache. Main memory 2008 may have stored therein control logic (i.e., computer software) and/or data. In some embodiments, main memory 2008 may include optical logic configured to perform sepsis detection, sepsis likelihood prediction, pathogen identification, and susceptibility testing, and generate recommendations for treatment of patients accordingly.

Computer system 2000 may also include one or more secondary storage devices or memory 2010. Secondary memory 2010 may include, for example, a hard disk drive 2012 and/or a removable storage drive 2014.

Removable storage drive 2014 may interact with a removable storage unit 2018. Removable storage unit 2018 may include a computer usable or readable storage device having stored thereon computer software (control logic) and/or data. Removable storage unit 2018 may be a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage drive 2014 may read from and/or write to removable storage unit 2018.

Secondary memory 2010 may include other means, devices, components, instrumentalities or other approaches for allowing computer programs and/or other instructions and/or data to be accessed by computer system 2000. Such means, devices, components, instrumentalities or other approaches may include, for example, a removable storage unit 2022 and an interface 2020. Examples of the removable storage unit 2022 and the interface 2020 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 2000 may further include a communication or network interface 2024. Communication interface 2024 may enable computer system 2000 to communicate and interact with any combination of external devices, external networks, external entities, etc. (individually and collectively referenced by reference number 2028). For example, communication interface 2024 may allow computer system 2000 to communicate with external or remote devices 2028 over communications path 2026, which may be wired and/or wireless (or a combination thereof), and which may include any combination of LANs, WANs, the Internet, etc. Control logic and/or data may be transmitted to and from computer system 2000 via communication path 2026.

Computer system 2000 may also be any of a personal digital assistant (PDA), desktop workstation, laptop or notebook computer, netbook, tablet, smartphone, smartwatch or other wearables, appliance, part of the Internet-of-Things, and/or embedded system, to name a few non-limiting examples, or any combination thereof.

Computer system 2000 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 2000 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 2000, main memory 2008, secondary memory 2010, and removable storage units 2018 and 2022, as well as tangible articles of manufacture embodying any combination of the foregoing. Such control logic, when executed by one or more data processing devices (such as computer system 2000), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 20. In particular, embodiments can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

Embodiments of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A method comprising:
receiving, by an analyzer device, a sample preparation cartridge and a sample container, the sample container containing a sample comprising pathogens;
installing a first needle from the sample preparation cartridge in a pipettor system in the analyzer device;
inserting the first needle into the sample container using the pipettor system;
transferring at least a portion of the sample from the sample container through the first needle to a processing tube in the sample preparation cartridge;
concentrating and enriching the pathogens of the transferred sample in the processing tube using the analyzer device, resulting in an enriched sample in the processing tube;
dispensing a plurality of aliquots of the enriched sample to a plurality of reaction wells in an antimicrobial susceptibility testing (AST) cartridge in the analyzer device, wherein each aliquot corresponds to a respective reaction well, and wherein each reaction well comprises an antimicrobial of a predetermined concentration;
incubating the aliquots in the reaction wells of the AST cartridge for a predetermined period of time for a reaction to occur between the pathogens in the aliquots and the antimicrobial in each reaction well;
acquiring an image of each reaction well in the AST cartridge by using a microscope in the analyzer device; and
determining, by a processor coupled to the microscope in the analyzer device, a susceptibility of the pathogens to the antimicrobial in each reaction well by analyzing the image.

2. The method of claim 1, wherein the sample container is a blood culture bottle in which the sample is incubated for an amount of time to allow the pathogens to grow but not reach a growth plateau.

3. The method of claim 1, wherein the sample container is a blood sample tube.

4. The method of claim 1, further comprising:
identifying a number of the pathogens in the transferred sample by using a fluorescent dye and the microscope in the analyzer device for labeling and counting the pathogens; and
in response to the identification, enriching or diluting the transferred sample to obtain a predetermined number of pathogens in the enriched sample.

5. The method of claim 1, wherein concentrating the pathogens of the transferred sample using the analyzer device comprises:
moving the processing tube to a centrifuge in the analyzer device;
centrifuging the processing tube in the centrifuge to concentrate the pathogens in the transferred sample; and
removing a fluid from the processing tube using the pipettor system, leaving the concentrated pathogens in the processing tube.

6. The method of claim 5, wherein the sample is a blood sample, and the method further comprises, prior to the centrifuging of the processing tube:
adding one or more lysis reagents to the processing tube; and
mixing the one or more lysis reagents with the blood sample in the processing tube to lyse blood cells in the blood sample.

7. The method of claim 6, wherein the one or more lysis reagents comprise one or more saponin-based buffers.

8. The method of claim 6, wherein the one or more lysis reagents comprise one or more detergents, surfactants, or proteases.

9. The method of claim 5, further comprising:
cleaning the concentrated pathogens after the additional predetermined period of time by centrifuging the processing tube and removing a supernatant from the processing tube to obtain the enriched sample.

10. The method of claim 5, wherein the processing tube further comprises magnetic beads configured to attach to the concentrated pathogens in the processing tube, the method further comprising:
retaining the concentrated pathogens attached to the magnetic beads in the processing tube by using a magnet station in the analyzer device; and
removing extraneous liquid from the processing tube, resulting in the enriched sample with the concentrated pathogens.

11. The method of claim 10, wherein the magnetic beads are coated with non-specific ligands.

12. The method of claim 10, wherein the magnetic beads are coated with specific ligands that are specific to a particular pathogen of the concentrated pathogens in the processing tube.

13. The method of claim 10, further comprising adding a treatment with proteases and/or DNAses after attaching the concentrated pathogens to the magnetic beads but prior to retaining the concentrated pathogens attached to the magnetic beads with the magnet.

14. The method of claim 5, further comprising:
adding one or more wash materials to the concentrated pathogens in the processing tube to clean and remove any blood components or debris from the concentrated pathogens, leaving the enriched sample in the processing tube.

15. The method of claim 14, wherein the one or more wash materials comprise a combination of one or more buffers, detergents, surfactants, and proteases.

16. The method of claim 1, further comprising:
adding the antimicrobial to each reaction well in the AST cartridge in a liquid form prior to the dispensing of the plurality of aliquots of the enriched sample to the reaction wells.

17. The method of claim 1, wherein the antimicrobial in each reaction well in the AST cartridge is in a dried or freeze-dried form prior to the dispensing the plurality of aliquots of the enriched sample to the reaction wells.

18. The method of claim 1, wherein the dispensing of the plurality of aliquots to the plurality of reaction wells comprises using jet dispensing to perform a contactless dispensing of the aliquots by a second needle in the sample preparation cartridge penetrating respective septums of the reaction wells and without the second needle contacting a bottom wall of each reaction well.

19. The method of claim 18, wherein the second needle is the same as the first needle.

20. The method of claim 18, wherein a volume of each aliquot dispensed by the second needle is in a range of about 0.5 microliters to about 10 microliters.

21. The method of claim 1, wherein a number of the pathogens in the sample is less than 200.

22. The method of claim 1, wherein a number of the pathogens in the enriched sample is in a range of about 1,000 to about 100,000.

23. The method of claim 1, wherein a number of the pathogens in the enriched sample is about 10,000.

24. The method of claim 1, wherein each reaction well comprises a bottom wall with an inner surface and an outer surface, wherein the reaction between the pathogens in the aliquots and the antimicrobial in each reaction well occurs above the inner surface of the bottom wall of each reaction well in the AST cartridge, and wherein the image of the AST cartridge is acquired at the outer surface of the bottom wall of each reaction well in the AST cartridge.

25. The method of claim 1, wherein the predetermined period of time for the incubation of the aliquots is about two hours or less.

26. The method of claim 1, further comprising:
after the incubating of the aliquots in each reaction well, moving the AST cartridge to a centrifuge in the analyzer device to move the pathogens in each aliquot to a bottom wall of each reaction well for acquiring the image of the AST cartridge.

27. The method of claim 1, further comprising:
after the incubating of the aliquots in each reaction well, using a magnet in the analyzer device to move the pathogens in each aliquot to a bottom wall of each reaction well for acquiring the image of the AST cartridge, wherein the pathogens are attached to magnetic beads in each aliquot.

28. The method of claim 1, further comprising:
adding a first fluorescent dye to each reaction well to stain the pathogens in the aliquots of the reaction wells prior to the acquiring of the image.

29. The method of claim 28, wherein the addition of the first fluorescent dye to each reaction well comprises using jet dispensing to perform a contactless dispensing of the first fluorescent dye by a second needle in the sample preparation cartridge penetrating respective septums of the reaction wells and without the second needle contacting a bottom wall of each reaction well.

30. The method of claim 29, wherein a volume of the first fluorescent dye dispensed to each reaction well by the second needle is in a range of about 0.5 microliters to 10 microliters.

31. The method of claim 30, wherein the second needle is the same as the first needle.

32. The method of claim 28, wherein the acquiring the image comprises acquiring one or more fluorescent images to detect the first fluorescent dye in each reaction well of the AST cartridge.

33. The method of claim 32, wherein the analyzing the image comprises computing a number of fluorescent pathogens in each reaction well in the AST cartridge.

34. The method of claim 28, further comprising:
adding a second fluorescent dye to each reaction well prior to the acquiring of the image.

35. The method of claim 34, wherein the first fluorescent dye comprises a DNA-binding dye that labels live cells in the aliquots of the reaction wells, and the second fluorescent dye comprises a fluorescent intercalating agent that labels dead cells, or that is not permeant to an intact cell membrane, in the aliquots of the reaction wells.

36. The method of claim 35, wherein determining the susceptibility of the pathogens comprises determining whether one or more of the pathogens are susceptible, intermediate, or resistant to the antimicrobial in each reaction well based on the staining of the cells in the aliquots by the first fluorescent dye and/or the second fluorescent dye.

37. The method of claim 36, wherein determining that a particular pathogen is resistant to a particular antimicrobial in a reaction well comprises:
determining that a ratio of a number of the dead cells to a number of the live cells is below a predefined threshold for the particular antimicrobial of a predetermined concentration based on the staining of the cells in the aliquots by the first fluorescent dye and/or the second fluorescent dye.

38. The method of claim 1, wherein determining the susceptibility of the pathogens comprises determining whether one or more of the pathogens are susceptible, intermediate, or resistant to the antimicrobial in each reaction well based on the staining of the cells in the aliquots by a first fluorescent dye.

39. The method of claim 1, wherein a first reaction well of the plurality of reaction wells comprises a first concentration of the antimicrobial, wherein the first concentration is a high dose that is higher than a clinical breakpoint for the antimicrobial and the pathogen.

40. The method of claim 39, wherein determining the susceptibility of the pathogens comprises comparing images of the first reaction well with a control to determine highly resistant pathogens in about one hour.

41. The method of claim 1, further comprising:
determining, by the processor, a minimum inhibitory concentration (MIC) of a particular antimicrobial for inhibiting growth of a particular pathogen in the one or more pathogens based on the image of the AST cartridge.

42. The method of claim 41, further comprising:
determining, by the processor, whether the particular pathogen is susceptible, intermediate, or resistant to the particular antimicrobial based on a value of the MIC of the particular antimicrobial by parsing a set of rules in a database communicatively coupled to the processor.

43. An antimicrobial susceptibility testing (AST) cartridge comprising:
a base comprising a plurality of reaction wells, each reaction well comprising a bottom wall, wherein the bottom wall is optically transparent;
a septum disposed over the base, the septum sealing each reaction well in the plurality of reaction wells, and
a cover disposed over the septum,
wherein each reaction well in the plurality of reaction wells contains an antimicrobial of a predetermined concentration for reacting with a respective aliquot of an enriched sample comprising pathogens, and wherein the antimicrobial is disposed within each reaction well.

44. The AST cartridge of claim 43, wherein the antimicrobial in each reaction well is in a liquid form.

45. The AST cartridge of claim 43, wherein the antimicrobial in each reaction well is in a dried or freeze-dried form.

46. The AST cartridge of claim 43, wherein the bottom wall of each reaction well in the plurality of reaction wells is configured for optical interrogation.

47. The AST cartridge of claim 43, wherein the bottom wall of each reaction well in the plurality of reaction wells is configured for fluorescence microscopy.

48. The AST cartridge of claim 43, wherein a diameter of the bottom wall of each reaction well is less than about 2 mm.

49. The AST cartridge of claim 43, wherein each reaction well comprises a conical shape.

50. The AST cartridge of claim 43, wherein each reaction well is configured to receive the aliquot of the enriched sample comprising pathogens by a contactless dispensing of the aliquot by a needle penetrating the septum of the reaction well without contacting the bottom surface of each reaction well.

51. The AST cartridge of claim 43, wherein the cover comprises a plurality of openings, wherein each opening aligns with a respective reaction well of the plurality of reaction wells in the base.

52. The AST cartridge of claim 43, wherein the plurality of reaction wells are configured to fit into corresponding wells in a temperature control block, wherein the temperature control block heats the plurality of reaction wells.

53. The AST cartridge of claim 43, wherein the base, the septum, and the cover each comprise an opening in a center of the AST cartridge, the opening being compatible for insertion by a pipettor for moving the AST cartridge.

54. The AST cartridge of claim 43, wherein the septum is overmolded into the cover to form a combined component, and wherein the combined component is assembled over the base by at least one of a snap-fit joint or a mechanical fastener.

55. The AST cartridge of claim 43, wherein the septum is a unibody that extends across the plurality of reaction wells.

56. The AST cartridge of claim 43, wherein the septum comprises multiple parts assembled together, wherein each part covers each reaction well.

57. The AST cartridge of claim 43, wherein the septum comprises at least one of a rubber, polytetrafluoroethylene (PTFE), thermoplastic elastomer (TPE), silicone, butyl rubber, or a combination thereof.

58. The AST cartridge of claim 43, wherein the septum comprises a double layer of polytetrafluoroethylene (PTFE) and another material selected from the group consisting of silicone, rubber, and butyl rubber.

59. The AST cartridge of claim 43, wherein the septum comprises a thickness of about 1 to 2 mm.

60. The AST cartridge of claim 43, wherein the plurality of reaction wells comprises about 100 reaction wells, wherein each reaction well holds a volume of about 30 microliters.

61. The AST cartridge of claim 43, wherein the cover is made of a polypropylene (PP) or a polycarbonate (PC) material.

62. The AST cartridge of claim 43, wherein the base is made of a polystyrene (PS) material.

63. The AST cartridge of claim 43, wherein the cover comprises an identifier that is scanned by an analyzer device for performing antimicrobial susceptibility testing.

64. The AST cartridge of claim 63, wherein the identifier is a data matrix or a barcode.

65. A system for enriching samples, comprising:
a housing configured to receive a sample container containing a sample comprising pathogens;
a pipettor system disposed inside the housing;
one or more centrifuges disposed inside the housing; and
a controller, wherein the controller is configured to:
transfer at least a portion of the sample from the sample container to a processing tube using the pipettor system;
centrifuge the processing tube using the one or more centrifuges to concentrate the pathogens in the transferred sample;
remove a fluid from the processing tube using the pipettor system, leaving the concentrated pathogens in the processing tube;
add a growth media to the concentrated pathogens in the processing tube using the pipettor system to grow the concentrated pathogens in the processing tube for a predetermined period of time; and
clean the concentrated pathogens after the predetermined period of time to obtain an enriched sample in the processing tube.

66. The system of claim 65, wherein the sample container is a blood culture bottle in which the sample is incubated for an amount of time to allow the pathogens to grow but not reach a growth plateau.

67. The system of claim 65, wherein the sample container is a blood sample tube.

68. The system of claim 65, further comprising a mixer disposed in the housing, wherein the sample is a blood sample, and wherein the controller is further configured to, prior to the centrifuging of the processing tube:
add, by using the pipettor system, one or more lysis reagents to the processing tube; and
use the mixer to mix the one or more lysis reagents with the blood sample in the processing tube to lyse blood cells in the blood sample.

69. The system of claim 68, wherein the one or more lysis reagents comprise one or more saponin-based buffers, detergents, surfactants, or proteases.

70. The system of claim 65, wherein the cleaning the concentrated pathogens comprises centrifuging the processing tube using the one or more centrifuges and removing a supernatant from the processing tube using the pipettor system to leave the enriched sample in the processing tube.

71. The system of claim 65, further comprising an antimicrobial susceptibility testing (AST) subsystem, wherein the processing tube further comprises magnetic beads configured to attach to the concentrated pathogens in the processing tube, and wherein the AST subsystem is configured to:
apply a magnetic force to the processing tube to retain the concentrated pathogens attached to the magnetic beads in the processing tube,
wherein the controller is further configured to remove extraneous liquid from the processing tube using the pipettor system, resulting in the enriched sample with the concentrated pathogens.

72. The system of claim 71, wherein the magnetic beads are coated with non-specific ligands.

73. The system of claim 71, wherein the magnetic beads are coated with specific ligands that are specific to a particular pathogen of the concentrated pathogens in the processing tube.

74. The system of claim 65, wherein the controller is further configured to:
add, using the pipettor system, one or more was materials to the concentrated pathogens in the processing tube to clean and remove any blood components or debris from the concentrated pathogens, leaving the enriched sample in the processing tube.

75. The system of claim 74, wherein the one or more wash materials comprise a combination of one or more buffers, detergents, surfactants, and proteases

76. The system of claim 65, wherein the sample container and the processing tube each comprise a septum allowing insertion by a first needle coupled to a first pipettor in the pipettor system.

77. The system of claim 76, wherein the transferring of at least a portion of the sample from the sample container to the processing tube comprises inserting the first needle through the septum of the processing tube and dispensing the at least a portion of the sample into the processing tube through the first needle.

78. The system of claim 65, further comprising:
a receptacle configured to receive an antimicrobial susceptibility testing (AST) cartridge containing a plurality of reaction wells; and
a microscope configured to acquire one or more images of the concentrated pathogens in the enriched sample after a transfer of the enriched sample to the plurality of reaction wells.

79. The system of claim 78, wherein the one or more centrifuges comprise:
a first centrifuge configured to hold and centrifuge the processing tube; and
a second centrifuge configured to hold and centrifuge the AST cartridge in a vertical orientation.

80. The system of claim 78, further comprising:
a magnet station configured to apply a magnetic force to move the concentrated pathogens to a bottom surface of the reaction wells in the AST cartridge, wherein the concentrated pathogens are attached to magnetic beads.

81. A system for analyzing samples, comprising:
a housing configured to receive a processing tube and an antimicrobial susceptibility testing (AST) cartridge, the AST cartridge comprising: a plurality of reaction wells, each reaction well comprising a bottom wall, wherein the bottom wall is optically transparent;
a heater disposed inside the housing;
a pipettor system disposed inside the housing;
a microscope disposed inside the housing; and
a controller, wherein the controller is configured to:
dispense, using the pipettor system, a plurality of aliquots of an enriched sample comprising pathogens from the processing tube to the plurality of reaction wells in the AST cartridge, wherein each aliquot corresponds to a respective reaction well, and wherein each reaction well comprises an antimicrobial of a predetermined concentration;
incubate, using the heater, the aliquots in the reaction wells of the AST cartridge for a predetermined period of time for a reaction to occur between the pathogens and the antimicrobial in each reaction well;
acquire, using the microscope, one or more images of a bottom wall of each reaction well in the AST cartridge; and
determine, by a processor coupled to the microscope, a susceptibility of the pathogens to the antimicrobial in each respective reaction well by analyzing the one or more images.

82. The system of claim 81, wherein the controller is further configured to:
add, using the pipettor system, a first fluorescent dye to each reaction well to stain pathogens in the aliquots of the reaction wells prior to the acquiring of the one or more images.

83. The system of claim 82, wherein the acquiring the one or more images comprises acquiring one or more fluorescent images to detect the first fluorescent dye in each reaction well of the AST cartridge.

84. The system of claim 83, wherein the analyzing the one or more images comprises computing a number of fluorescent pathogens in each reaction well in the AST cartridge.

85. The system of claim 82, wherein the controller is further configured to:
add, using the pipettor system, a second fluorescent dye to each reaction well prior to the acquiring of the one or more images.

86. The system of claim 85, wherein the first fluorescent dye comprises a DNA-binding dye that labels live cells in the aliquots of the reaction wells, and the second fluorescent dye comprises a fluorescent intercalating agent that labels dead cells in the aliquots of the reaction wells.

87. A method of manufacturing an antimicrobial susceptibility testing (AST) cartridge, the method comprising:
fabricating a cover comprising a plurality of openings;
overmolding a septum into the cover, wherein a first side of the septum extends across the plurality of openings;
producing a base comprising a plurality of reaction wells; and
attaching the base to a second side of the septum, wherein the second side of the septum extends across and seals the plurality of reaction wells in the base.

88. The method of claim 87, wherein the cover comprises polypropylene (PP) or polycarbonate (PC).

89. The method of claim 87, wherein the base comprises polystyrene (PS).

90. The method of claim 87, further comprising:
prior to attaching the base to the second side of the septum, adding an antimicrobial of a predetermined concentration in a liquid form to each reaction well; and
drying the antimicrobial in each reaction well to a dried or a freeze-dried form using forced air.

91. The method of claim 90, wherein the adding of the antimicrobial and the drying of the antimicrobial in each reaction well is completed within a predetermined period of time to prevent degradation of the antimicrobial.

92. The method of claim 91, wherein the predetermined period of time comprises about 15 minutes.

93. The method of claim 91, wherein the predetermined period of time comprises about 10 minutes.

94. The method of claim 87, wherein fabricating the cover comprises using injection molding.

95. The method of claim 87, wherein attaching the base to the second side of the septum comprises using at least one of a snap-fit joint or a mechanical fastener.

96. The method of claim 87, further comprising:
aligning each opening in the cover with a respective reaction well of the plurality of reaction wells in the base during the attaching of the base to the second side of the septum.

97. The method of claim 87, wherein producing the base comprises producing the plurality of reaction wells connected together as a single component.

98. The method of claim 87, wherein the septum comprises at least one of a rubber, polytetrafluoroethylene (PTFE), thermoplastic elastomer (TPE), silicone, butyl rubber, or a combination thereof.

99. The method of claim 87, wherein the septum comprises a double layer of polytetrafluoroethylene (PTFE) and another material selected from the group consisting of silicone, rubber, and butyl rubber.
